(19) United States
(12) Patent Application Publication
(10) Pub. No.: US 2023/0110028 A1
(43) Pub. Date: Apr. 13, 2023

(54) OXYBATE POLYETHYLENE GLYCOL PRODRUGS

(71) Applicant: Tris Pharma, Inc., Monmouth Junction, NJ (US)

(72) Inventor: Paras Rameshlal Jain, Dayton, NJ (US)

(21) Appl. No.: 17/818,733

(22) Filed: Aug. 10, 2022

Related U.S. Application Data
(60) Provisional application No. 63/231,878, filed on Aug. 11, 2021.

(51) Int. Cl. A61K 47/60; A61K 47/69; A61K 31/19

(52) U.S. Cl. CPC A61K 47/60; A61K 47/6951; A61K 31/19

(57) ABSTRACT

Oxybate-PEG ester prodrugs are provided herein in which the polyethylene glycol is bound to oxybate via an ester linkage. These oxybate-PEG esters may be further bound to a cyclodextrin or an anion exchange resin, via an ester linkage between the oxybate and the cyclodextrin or via ionic bonding between oxybate the anion exchange resin. Compounds and compositions containing these compounds are useful in immediate release and modified release compositions for treating narcolepsy and other disorders.

### CROSS-REFERENCE TO RELATED APPLICATION

This application claims the benefit under 37 CFR 119(e) of prior provisional patent application U.S. 63/231,878, filed Aug. 11, 2021, which is hereby incorporated by reference in its entirety.

### BACKGROUND OF THE INVENTION

Sodium oxybate is a central nervous system (CNS) depressant indicated for the treatment of cataplexy or excessive daytime sleepiness (EDS) in patients 7 years of age and older with narcolepsy. Sodium oxybate is currently commercially available in a solution 500 mg/ml, under the brand name XYREM® (Jazz Pharmaceuticals), at a dose of 4.5 gm to 9 gm taken in two divided doses. See, XYREM® Product label. The first dose is taken before bed and the second dose is taken 2.5-4 hours post first dose. Thus, patient need to wake up in the night and administer the second dose. This is not convenient for patients, resulting in poor patient compliance. Due to high sodium content per unit dose, sodium oxybate needs to be monitored vary carefully in patients sensitive to high sodium intake (for example patients with heart failure, hypertension, or renal impairment). Further, since oxybate has very short elimination half-life (e.g., about 30-40 min), a high dose needs to be administered in order to maintain effective plasma drug levels covering entire dosing interval.

There is a risk of toxicity related to high Cmax of sodium oxybate, where sodium is reported to enhance rate of oxybate absorption. This risk of toxicity increases when taken with alcohol since both follow common metabolic pathway and competition in metabolism could increase the Cmax of oxybate, increasing the risk of respiratory depression.

Oxybate exhibits site specific absorption, faster and greater absorption from upper parts of gastro-intestinal tract and slower and lower absorption from lower parts of gastro-intestinal tract. GHB is substrate for monocarboxylic acid transporters (MCTs). Although MCTs are present along the entire GI tract; GHB absorption is likely to be faster and greater in upper part of GIT due to higher proton gradient. To ensure rapid absorption, it is important that solubility of GHB salts is sufficient in GI fluid present in the lumen of upper GI tract. Instant liberation of free acid GHB from salt in acidic media could result in precipitation of GHB. Only the sodium salt of oxybate has enough solubility in acidic media, but the use of sodium salt is not advised in high risk patients. Sodium appears to play role in faster absorption via sodium dependent MCTs. As a result, development of once a night long acting formulation of oxybate becomes challenging.

Oxybate exhibits saturable elimination via metabolism and there is lack of dose linearity, wherein AUC and Cmax increase more than proportionality in XYREM® reference product dose increases from 4.5 g to 9 g per night. This increases risk of Cmax crossing the therapeutic index and increasing risk of toxicity, including severe respiratory depression. Sodium oxybate is unstable in acidic medium and forms gamma-butyrolactone (GBL) which is impurity. Sodium oxybate tends to undergo conversion to gamma-butyrolactone (GBL) at pH values below 7, with the lower the pH, the faster the conversion to GBL. Although this conversion to GBL is reversible, conversion of GBL back to GHB is very slow at pH<4.5 (e.g., pH of stomach). Sodium oxybate is highly hygroscopic and hence poses difficulties in handling and processing. Due to short elimination half-life and rapid clearance from plasma, sodium oxybate has been reported to be abused and misused in crimes like “rape”, as it is difficult to detect in plasma after few hours. See, e.g., Wing Ki Lam, Melanie A. Felmlee, and Marilyn E. Morris Monocarboxylate Transporter-Mediated Transport of gamma-Hydroxybutyric Acid in Human Intestinal Caco-2 Cells DRUG METABOLISM AND DISPOSITION Vol. 38, No. 3, 2010, pages 441-447.

All these limitations result in poor patient compliance. Inadequacies of current drug delivery options to tackle these problems associated with use of sodium oxybate result in poor management of narcolepsy and cataplexy creating unmet medical need.

### BRIEF DESCRIPTION OF THE FIGURES

FIGS. 1A-1C shows the structure and conformation of natural cyclodextrins, alpha, beta and gamma.

### SUMMARY OF THE INVENTION

Provided herein are oxybate-polyethylene glycol (PEG) ester prodrugs and conjugates.

In certain embodiments, the compounds are oxybate-PEG-cyclodextrin double cleavage prodrugs and/or conjugates, wherein the said double cleavage prodrugs contain ester linkage between the hydroxyl group of oxybate and carboxylic acid group of PEG acid and another ester linkage between carboxylic acid group of oxybate and hydroxyl group of cyclodextrin.

Further provided are double cleavage prodrugs and/or conjugates based on esterification and anion exchange reactions wherein the said double cleavage prodrugs contain ester linkage between hydroxyl group of oxybate and carboxylic acid group of PEG acid and; ionic bond between ionized carboxylic acid group of oxybate and cationic quaternary nitrogen of strong base type anion exchange resin.

In certain embodiments an polyethylene glycol (PEG)-oxybate ester prodrug or conjugate is provided which comprises:
(A) an oxybate-PEG ester having the structure: PEG-O—CH₂—COO—CH₂—CH₂—CH₂—COOH, wherein PEG is bound to oxybate through an ester linkage formed between the gamma hydroxyl group of oxybate and carboxylic acid group of an Alkoxy-PEG acid (e.g., monomethoxy PEG);
(B) a PEG-oxybate-cyclodextrin di-ester, wherein PEG is bound to oxybate through an ester linkage formed between the gamma hydroxyl group of oxybate and carboxylic acid group of an Alkoxy-PEG acid (e.g., monomethoxy PEG acid) and cyclodextrin is bound to the oxybate through another ester linkage formed between carboxylic acid group of oxybate and hydroxyl group of cyclodextrin; or
(C) a PEG-oxybate-anion exchange resin complex, wherein PEG is bound to oxybate through an ester linkage formed between the gamma hydroxyl group of oxybate and carboxylic acid group of alkoxy PEG acid (e.g., monomethoxy PEG acid) and the anion exchange resin is bound through ionic bond formed between quaternary ammonium group of the resin and carboxylic acid group of oxybate, the complex is optionally in a polymeric matrix, and/or optionally coated.

In certain embodiments, a pharmaceutical composition is provided which comprises:
(A) one or more oxybate-PEG esters which may be the same or different and which have the structure: PEG-O—CH₂—COO—CH₂—CH₂—CH₂—COOH, wherein the polyethylene glycol is bound to the oxybate through an ester linkage;
(B) one or more PEG-oxybate-cyclodextrin di-ester, wherein PEG is bound to oxybate through an ester linkage formed between the gamma hydroxyl group of oxybate and carboxylic acid group of an Alkoxy-PEG acid (e.g., monomethoxy PEG) and cyclodextrin is bound to the oxybate through another ester linkage formed between carboxylic acid group of oxybate and hydroxyl group of cyclodextrin; or
(C) one or more PEG-oxybate-anion exchange resin complex, wherein PEG is bound to oxybate through an ester linkage formed between the gamma hydroxyl group of oxybate and carboxylic acid group of PEG acid and the anion exchange resin is bound through ionic bond formed between quaternary ammonium group of the resin and carboxylic acid group of oxybate, the complex is optionally in a polymeric matrix, and/or optionally coated.

In certain embodiments, the composition comprises an admixture of two or more different oxybate-PEG ester compounds. Such compounds may comprise a mixture of oxybate PEG esters, wherein the PEG has different molecular weights and/or other differences in the oxybate-PEG esters.

In certain embodiments, the composition comprises an amount of oxybate equivalent of 2.25 g to 12 g sodium oxybate, or 4.5 gm sodium oxybate to 9 gm sodium oxybate.

In certain embodiments, the pharmaceutical composition is a powder-for-suspension or powder-for-solution formulated for oral dosing. In certain embodiments, the pharmaceutical composition is an aqueous suspension or solution formulated for injection.

In certain embodiments, the pharmaceutical composition is sodium-free.

In certain embodiments, the pharmaceutical composition comprises one or more PEG-oxybate alpha-cyclodextrin esters, one or more PEG-oxybate beta-cyclodextrin esters, and/or one or more oxybate-PEG gamma-cyclodextrin ester compounds is in a modified release form.

In certain embodiments, the pharmaceutical composition comprises one or more PEG-oxybate alpha-cyclodextrin esters, one or more PEG-oxybate beta-cyclodextrin di-esters, and/or one or more PEG-oxybate gamma-cyclodextrin ester compounds is in an immediate release form.

In certain embodiments, the pharmaceutical composition comprises an PEG-oxybate-anion exchange resin complex prodrug and/or conjugate in an immediate release form, a modified release form, or combinations thereof. Optionally such a composition may contain one or more active form of oxybate and/or one or more other oxybate prodrugs and/or conjugates.

In certain embodiments, a pharmaceutical composition as provided herein, further comprises one or more pharmaceutically acceptable oxybate salts or a gamma hydroxybutyrate prodrug and/or conjugate.

In certain embodiments, the pharmaceutical composition comprises one or more oxybate cyclodextrin compounds in both immediate release and extended release forms.

In certain embodiments, a method for improving the abuse-resistance of an oxybate composition comprising generating oxybate-PEG cyclodextrin esters as provided herein.

These and other advantages of the invention will be apparent from the following detailed description of the invention.

### DETAILED DESCRIPTION OF THE INVENTION

The oxybate-PEG esters and compositions containing same provided herein are useful for a variety of purposes, including improved methods of delivery and treatment of patients with oxybate, e.g., for narcolepsy and cataplexy. In certain embodiments, a method for reducing or eliminating sodium content in oxybate compositions are provided. Also provides are compositions which are more abuse-resistant than oxybate salts and other compositions.

Covalent conjugates of oxybate and PEG are provided herein and are useful in pharmaceutical compositions. In certain embodiments, one or more oxybate PEG esters is an oxybate prodrug. Such a “prodrug” is a biologically inactive oxybate PEG ester is a biologically inactive compound which is metabolized in the body to produce the active oxybate compound. In certain embodiments, one or more of the prodrugs provided herein may be optionally combined with an oxybate, oxybate salt (e.g., sodium oxybate), another active compound, or mixtures thereof. Optionally, a composition may contain an admixture of an oxybate PEG ester prodrug and one or more different oxybate PEG esters.

As used herein, the term “oxybate PEG ester” encompasses the oxybate-PEG mono-esters, di-esters, or polymeric esters, oxybate-PEG cyclodextrin esters, and oxybate-PEG-anion exchange resin complexes (i.e., oxybate-PEG resinates) unless otherwise specified. Unless the type of cyclodextrin is specified, it will be understood that this term encompasses any natural or synthetic cyclodextrin.

In certain embodiments, the oxybate-PEG esters, di-esters, or PEG-oxybate-anion exchange resin complexes provided herein are sodium free. In certain embodiments, these compounds are also formulated in compositions which are substantially free of sodium. Thus, in these embodiments, these compounds are suitable for patients having sodium intake restrictions.

Without wishing to be bound by theory, certain of these compounds are prodrugs which are biologically inactive until bio-activated to release the biologically active oxybate moiety. In certain embodiments, bio-activation involves enzymatic hydrolysis by esterase enzymes present in GI tract and/or liver. This may become a rate determining step (RDS) and would help in prolonging/delaying the elimination of oxybate. Thus, in certain embodiments, the oxybate PEG esters may provide oxybate with an increased half-life. Without wishing to be bound by theory, since bio-activation is a RDS, liver enzymes would not become saturated even at higher dose and hence non-linear increase in Cmax would not be observed. This reduces the risk of potentially serious Cmax dependent side effects of oxybate currently observed for sodium oxybate products. Further, bio-activation is expected to be a slow process, permitting detection of oxybate in plasma would be possible for prolonged time periods. Thus, the compounds and compositions provided herein prevent misuse and diversion and lower abuse potential is expected. GBL formation from oxybate involves intra-molecular cyclization by interaction between hydroxyl and carboxylic acid groups of oxybate. Since oxybate in the oxybate-PEG ester prodrugs do not have free hydroxyl groups; such prodrugs would be resistant to GBL formation and would have improved chemical stability, both in solid state and in dissolved state. Similarly, since oxybate in the PEG-oxybate cyclodextrin-di ester prodrugs do not have free hydroxyl and carboxylic acid groups; such prodrugs would be resistant to GBL formation and would have improved chemical stability, both in solid state and in dissolved state.

Compositions provided herein are useful in treating conditions for which oxybate is approved, including, narcolepsy and cataplexy by administering to patients and by achieving one or more of the following advantages over sodium oxybate: reduce sodium such that product would be suitable for use in patients sensitive to high sodium intake, increased elimination half-life supporting once a night dosage regimen providing long acting product; reduce the rate of absorption thereby reducing the risk of respiratory depression associated with higher Cmax, improved patient compliance, increase safety and reduce toxicity, prevent/reduce abuse and misuse liability, increase physico-chemical stability, and reduce

### Oxybate

The oxybate esters provided herein are prepared using the active drug gamma hydroxybutyrate (GHB) or oxybate, or a pharmaceutically acceptable salt thereof. Suitable pharmaceutically acceptable salts of oxybate (e.g., a magnesium oxybate), and mixtures thereof. Suitable salts of oxybate or GHB include, e.g., the calcium, lithium, potassium, sodium and magnesium salts. In certain embodiments, the sodium salt (sodium oxybate) is not present in the final pharmaceutical composition (final product) containing the oxybate ester. In certain embodiments, the magnesium salt of oxybate is not present in the final pharmaceutical composition. In certain embodiments, an oxybate salt is selected for use as a starting material, e.g., from a potassium, magnesium, or calcium salt, or mixtures thereof. Representative salts are also described in US 2012/0076865, incorporated by reference herein. Sodium oxybate (gamma-hydroxybutyrate (GHB)) is currently available from Jazz Pharmaceuticals, Inc. as Xyrem® oral solution. Sodium oxybate is a white to off-white, crystalline powder that is very soluble in aqueous solutions. Methods of making GHB salts are described, for example, in U.S. Pat. No. 4,393,236, the disclosure of which is incorporated herein by reference.

### Polyethylene Glycol

Pharmaceutical grades of poly (ethylene glycol) (PEG) may be purchased commercially for use in preparing the compounds provided herein. Alternatively, PEG compounds may be synthesized using published methods.

PEG is a unique polyether diol, usually manufactured by aqueous anionic polymerization of ethylene oxide. Initiation of ethylene oxide polymerization using anhydrous alkanols such as methanol or derivatives including methoxyethyl ethanol results in a mono-alkoxy capped poly (ethylene glycol) such as methoxy PEG (mPEG). The polymerization reaction can modulated and a variety of molecular weights (1000-50000) can be obtained with low polydispersities. These polymers are amphiphilic and dissolve in organic solvents as well as in water. See, e.g., Richard B. Greenwald, Yun H. Choe, Jeffrey McGuire, Charles D. Conover Effective drug delivery by PEGylated drug conjugates Advanced Drug Delivery Reviews 55 (2003) 217-250; Shashwat S. Banerjee, Naval Aher, Rajesh Patil, and Jayant Khandare Poly(ethylene glycol)-Prodrug Conjugates: Concept, Design, and Applications Journal of Drug Delivery Volume 2012, Article ID 103973, 17 pages. PEGylation is the process of attaching the strands of the polymer PEG to molecules. Any suitable Veronese, F. M.; Harris, J. M. (2002). “Introduction and overview of peptide and protein pegylation”. Advanced Drug Delivery Reviews. 54 (4): 453-456.] [Porfiryeva, N. N.; Moustafine, R. I.; Khutoryanskiy, V. V. (2020 Jan. 1). “PEGylated Systems in Pharmaceutics”. Polymer Science, Series C. 62 (1): 62-74.]. [Milla, P; Dosio, F (13 Jan. 2012). “PEGylation of proteins and liposomes: a powerful and flexible strategy to improve the drug delivery”. Current Drug Metabolism. 13 (1): 105-119].

PEG is a polyethylene glycol residue having the following structure:

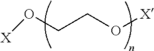

wherein n is an integer of 0-25; wherein X′ is OCHR—COOH, wherein R is H or C1-C4-alkyl (CH₃, CH₂CH₃, CH₂CH₂CH₃, CH₂CH₂CH₂CH₃) and X is OCH3, OCH2CH3, OCH2CH2CH3, OCH2CH2CH2CH3, COOH. In certain embodiments, R is a methyl group (—CH₂—), ethyl group (CH₂CH₃), propyl(CH₂CH₂CH₃) group, or butyl group CH₂CH₂CH₂CH₃)). In certain embodiments, R is a methyl group.

In certain embodiments, the PEG as a molecular weight of about 1000 to about 50,000, or about 2500 to about 25,000, or about 5000 to about 15,000, or values therebetween are selected. Molecular weight may be determined using any suitable method, e.g., a rheological method. Ester prodrugs (single cleavage type):

### Reaction Between Oxybate Salt and PEG Acid

HO—CH₂—CH₂—CH₂—COO⁻Na⁺+mPEG-OCH₂—COOH→mPEG-O—CH₂—COO—CH₂—CH₂—CH₂—COO⁻H⁺+H₂O Single cleavage prodrug of oxybate

### Cyclodextrins

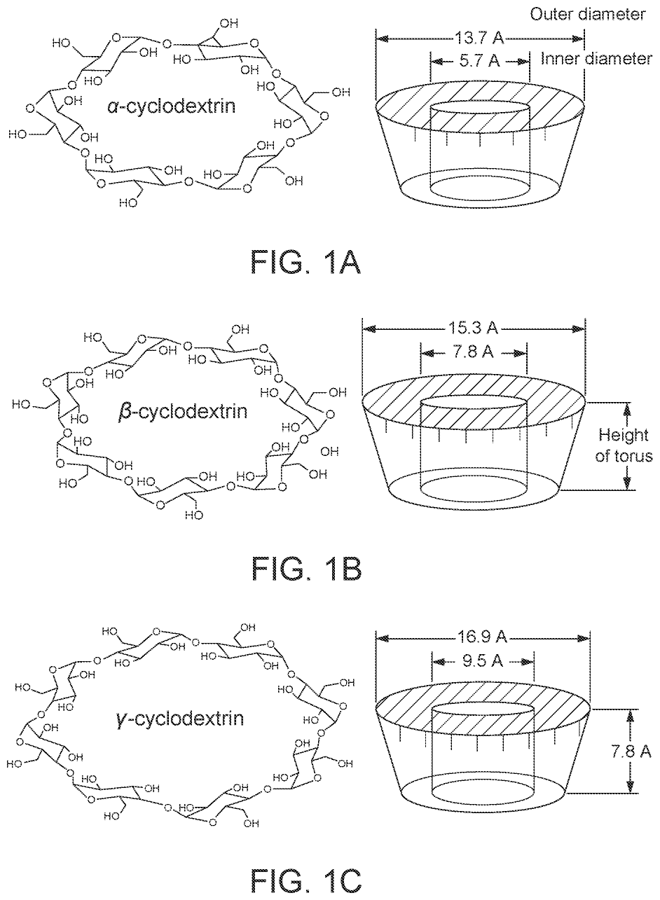

As used herein “cyclodextrins” refers to chemically and physically stable macromolecules produced by enzymatic degradation of starch. They are water-soluble, biocompatible in nature with hydrophilic outer surface and lipophilic cavity. They have the shape of truncated cone or torus rather than perfect cylinder because of the chair conformation of glucopyranose unit. See, e.g., Bina Gidwani and Amber Vyas A Comprehensive Review on Cyclodextrin-Based Carriers for Delivery of Chemotherapeutic Cytotoxic Anticancer Drugs BioMed Research International Volume 2015, Article ID 198268, 15 pages]. See e.g., FIGS. 1A-1C. Cyclodextrins are classified as natural and derived cyclodextrins. Natural cyclodextrins comprise three well known industrially produced (major and minor) cyclic oligosaccharides. The most common natural cyclodextrins are α, β, and γ consisting of 6, 7, and 8 glucopyranose units, respectively. They are generally crystalline, homogeneous, and non-hygroscopic substances. FIGS. 1A-1C shows the structure and conformation of natural cyclodextrins. Various hydrophilic, hydrophobic, and ionic derivatives have been developed. Polymerized cyclodextrins are high molecular weight compounds, either water soluble or insoluble. The examples of polymerized cyclodextrins are soluble anionic β-cyclodextrin polymer, soluble γ-cyclodextrin polymer, and epichlorohydrin β-cyclodextrin polymer. The table lists certain characteristic features and properties of useful cyclodextrins:

| | | |
|---|---|---|
| | Solubility | Mol. |
| Name of cyclodextrin | (mg/mL) | Wt. (Da) |

Natural cyclodextrins

| | | |
|---|---|---|
| Alpha cyclodextrin | 145 | 972 |
| Beta cyclodextrin | 18.5 | 1135 |
| Gamma cyclodextrin | 232 | 1297 |

Chemically modified cyclodextrins

| | | |
|---|---|---|
| Hydroxypropyl-β-cyclodextrin | ≥600 | 1400 |
| Sulfobutyl ether-β-cyclodextrin | ≥500 | 2163 |
| Randomly methylated-β-cyclodextrin | ≥500 | 1312 |
| Hydroxypropyl-γ-cyclodextrin | ≥500 | 1576 |

Polymerized cyclodextrins

| | | |
|---|---|---|
| Epichlorohydrin-β-cyclodextrin | >500 | 112000 |
| Carboxy methyl epichlorohydrin | >250 | 2000000- |
| beta cyclodextrin | | 15000000 |

While the examples herein illustrate esters based on natural cyclodextrins, it will be understood that synthetic cyclodextrins may be substituted therefor.

In certain embodiments, a PEG-oxybate cyclodextrin di-ester is provided in which one or more PEG-oxybate moieties is covalently bound to an alpha-cyclodextrin backbone (e.g., through a primary or secondary hydroxy group of a glucose ring). The cyclodextrin may be bound to at least one oxybate-PEG with the point of as follows: —COCH₂CH₂CH₂O—CO—CH₂O-PEG

to form an PEG-oxybate alpha-cyclodextrin di-ester and optionally may also contain a non-PEGylated oxybate with the point of attachment as follows: (—COCH₂CH₂CH₂OH).

In one embodiment, an oxybate alpha-cyclodextrin di-ester has the structure of Formula IA:

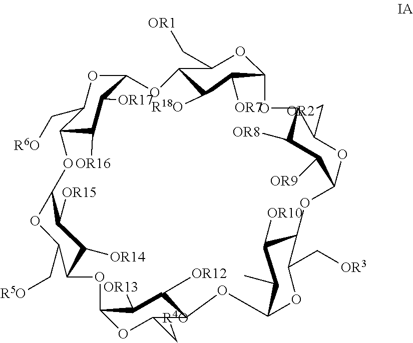

wherein in Formula IA, R¹-R⁸ are independently selected from —H, —COCH₂CH₂CH₂OH or —COCH₂CH₂CH₂O—CO—CH₂O-PEG to form an PEG-oxybate alpha-cyclodextrin di-ester, provided that at least one of R¹-R¹⁸ is —COCH₂CH₂CH₂O—CO—CH₂O-PEG.

In certain embodiments, when synthesized, a PEG-oxybate alpha-cyclodextrin ester produced as a mixture of one to eighteen, one to six, two, or more PEG-oxybate alpha-cyclodextrin ester compounds having a structure of Formula IA. In certain embodiments, a single PEG-oxybate alpha-cyclodextrin ester is used in a pharmaceutical composition. In other embodiments, a mixture of two or more different PEG-PEG-oxybate alpha-cyclodextrin esters are selected for use in a pharmaceutical composition.

In certain embodiments, a pharmaceutical composition comprises an admixture of one to six different PEG-oxybate alpha-cyclodextrin ester compounds, which contain at least one oxybate-PEG. Optionally, these compounds may be differ in the type of PEG (e.g., Alkoxy linkage and/or molecular weight) and/or in the number of oxybate linkages.

In certain embodiments, compounds and compositions are provided in which one or more oxybate moieties is covalently bound to an beta-cyclodextrin backbone (e.g., through a or secondary hydroxy group of a glucose ring). The beta-cyclodextrin may be bound to at least one oxybate-PEG with the point of as follows: —COCH₂CH₂CH₂O—CO—CH₂O-PEG to form a -PEG-oxybate beta-cyclodextrin di-ester and optionally may also contain a non-PEGylated oxybate with the point of attachment as follows: —COCH₂CH₂CH₂OH.

In one embodiment, a PEG-oxybate beta-cyclodextrin di-ester has the structure of Formula IB:

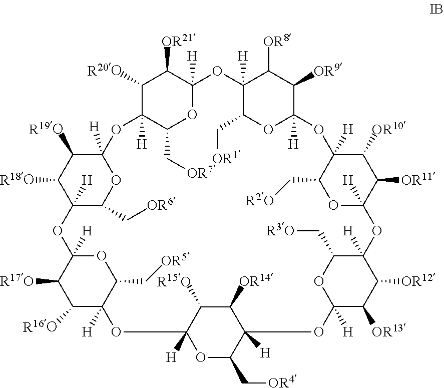

wherein in Formula IB, R^{1′}-R^{21′} are independently —H, or —COCH₂CH₂CH₂O—CO—CH₂O-PEG to form an PEG oxybate beta-cyclodextrin di-ester, provided that at least one of R′-R^{7′} or at least one of R^{8′}-R^{21′} is PEG-oxybate beta-cyclodextrin di-ester.

In certain embodiments, the PEG-oxybate beta-cyclodextrin di-ester has the structure of Formula IB and wherein two or more of R^{1′}-R^{21′} are —COCH₂CH₂CH₂OH or —COCH₂CH₂CH₂O—CO—CH₂O-PEG.

In certain embodiments, when synthesized, a -PEG-oxybate beta-cyclodextrin di-ester produced as a mixture of one to twenty-one, one to seven, two to twenty-one, and various combinations of PEG-oxybate beta-cyclodextrin di-ester compounds having a structure of Formula IB. In certain embodiments, a single PEG-oxybate beta-cyclodextrin diester is used in a pharmaceutical composition. In other embodiments, a mixture of two or more different PEG-oxybate-beta-cyclodextrin di-esters are selected for use in a pharmaceutical composition. In certain embodiments, a pharmaceutical composition comprises an admixture of one to seven different PEG-oxybate beta-cyclodextrin diester compounds. Optionally, these compounds may be differ in the type of PEG (e.g., Alkoxy linkage and/or molecular weight) and/or in the number of oxybate linkages.

In certain embodiments, compounds and compositions are provided in which one or more oxybate-PEG moieties is covalently bound to an gamma-cyclodextrin backbone (e.g., through a or secondary hydroxy group of a glucose ring). The cyclodextrin may be bound to at least one oxybate-PEG with the point of as follows: —COCH₂CH₂CH₂O—CO—CH₂O-PEG to form an oxybate-PEG gamma-cyclodextrin di-ester and optionally may also contain a non-PEGylated oxybate with the point of attachment as follows: —COCH₂CH₂CH₂OH. In certain embodiments, the oxybate-PEG cyclodextrin is an oxybate-PEG ester of Formula IC:

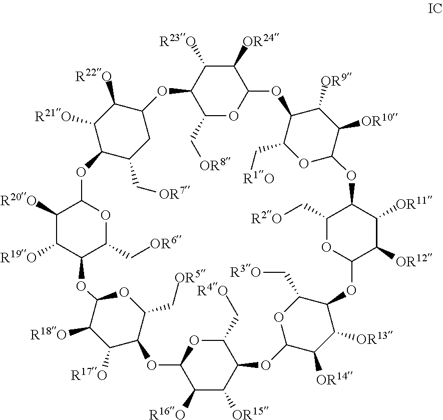

wherein in Formula IC, R^{1″}-R^{24″} are independently selected from —H, or —COCH₂CH₂CH₂O—CO—CH₂O-PEG

to form a gamma-cyclodextrin oxybate di-ester,
provided that at least one of R^{1″}-R^{24″} is COCH₂CH₂CH₂O—CO—CH₂O-PEG.

In certain embodiments, when synthesized, an oxybate-PEG gamma-cyclodextrin ester produced as a mixture of one, two or more PEG-oxybate gamma-cyclodextrin esters having a structure of Formula IC. In certain embodiments, a single PEG-oxybate gamma-cyclodextrin ester is used in a pharmaceutical composition. In other embodiments, a mixture of two or more different PEG-oxybate gamma-cyclodextrinesters are selected for use in a pharmaceutical composition. In certain embodiments, a pharmaceutical composition comprises an admixture of PEG-oxybate gamma-cyclodextrin ester compounds.

These oxybate cyclodextrin esters may be used alone, or in combination or admixture with one another, to prepare a pharmaceutical composition.

Oxybate-PEG ester prodrugs and uses thereof are provided in multiple embodiments.

In certain embodiments, PEG-oxybate beta cyclodextrin double cleavage prodrugs are provided. These double cleavage prodrugs contain ester linkage between hydroxyl group of oxybate and carboxylic acid group of PEG acid and; another ester linkage between carboxylic acid group of oxybate and one hydroxyl group of beta-cyclodextrin.

In certain embodiments, PEG-oxybate beta cyclodextrin double cleavage prodrugs are provided which contain an ester linkage between hydroxyl group of oxybate and carboxylic acid group of PEG acid and another ester linkage between carboxylic acid groups of two oxybate single cleavage prodrugs and two hydroxyl groups of beta-cyclodextrin.

In certain embodiments, PEG-oxybate beta cyclodextrin double cleavage prodrugs are provided which contain and ester linkage between hydroxyl group of oxybate and carboxylic acid group of PEG acid and another ester linkage between carboxylic acid groups of three oxybate single cleavage prodrugs and three hydroxyl groups of beta-cyclodextrin.

In certain embodiments, PEG-oxybate beta cyclodextrin double cleavage prodrugs are provided which contain an ester linkage between hydroxyl group of oxybate and carboxylic acid group of PEG acid and another ester linkage between carboxylic acid groups of four oxybate single cleavage prodrugs and four hydroxyl groups of beta-cyclodextrin.

In certain embodiments, PEG-oxybate beta cyclodextrin double cleavage prodrugs are provided which contain an ester linkage between the hydroxyl group of oxybate and the carboxylic acid group of PEG acid and another ester linkage between carboxylic acid groups of the five oxybate single cleavage prodrugs and the five hydroxyl groups of beta-cyclodextrin.

In still other embodiments, PEG-oxybate beta cyclodextrin double cleavage prodrugs are provided in which the double cleavage prodrugs contain an ester linkage between the hydroxyl group of oxybate and the carboxylic acid group of PEG acid and another ester linkage between the carboxylic acid groups of six oxybate single cleavage prodrugs and six hydroxyl groups of beta-cyclodextrin.

In certain embodiments, PEG-oxybate beta cyclodextrin double cleavage prodrugs are provided which contain ester linkage between the hydroxyl group of oxybate and the carboxylic acid group of PEG acid and another ester linkage between the carboxylic acid groups of seven oxybate single cleavage prodrugs and seven hydroxyl groups of beta-cyclodextrin.

In yet another embodiment, PEG-oxybatealpha-cyclodextrin double cleavage prodrugs are provided which contain an ester linkage between hydroxyl group of oxybate and carboxylic acid group of PEG acid and another ester linkage between a carboxylic acid group of oxybate and a hydroxyl group of alpha-cyclodextrin.

In yet another embodiment, PEG-oxybatealphacyclodextrin double cleavage prodrugs are provided which an ester linkage between hydroxyl group of oxybate and carboxylic acid group of PEG acid and another ester linkage between two carboxylic acid groups of oxybate single cleavage prodrugs and two hydroxyl groups of alpha-cyclodextrin.

In yet another embodiment, oxybate-PEG-alpha cyclodextrin double cleavage prodrugs are provided wherein the said double cleavage prodrugs contain an ester linkage between hydroxyl group of oxybate and carboxylic acid group of PEG acid and another ester linkage between three carboxylic acid groups of oxybate single cleavage prodrugs and three hydroxyl groups of alpha-cyclodextrin.

In yet another embodiment, PEG-oxybate-alpha cyclodextrin double cleavage prodrugs are provided which contain ester linkage between hydroxyl group of oxybate and carboxylic acid group of PEG acid and; another ester linkage between four carboxylic acid groups of oxybate single cleavage prodrugs and four hydroxyl groups of alpha-cyclodextrin.

In yet another embodiment, PEG-oxybate-alpha cyclodextrin double cleavage prodrugs are provided which contain an ester linkage between hydroxyl group of oxybate and carboxylic acid group of PEG acid and another ester linkage between five carboxylic acid groups of oxybate single cleavage prodrugs and five hydroxyl groups of alpha-cyclodextrin.

In yet another embodiment, PEG-oxybate-alpha cyclodextrin double cleavage prodrugs are provide which contain an ester linkage between hydroxyl group of oxybate and carboxylic acid group of PEG acid and another ester linkage between six carboxylic acid groups of oxybate single cleavage prodrugs and six hydroxyl groups of alpha-cyclodextrin.

In yet another embodiment, PEG-oxybate-gamma cyclodextrin double cleavage prodrugs are provide which contain an ester linkage between a hydroxyl group of oxybate and carboxylic acid group of PEG acid and another ester linkage between a carboxylic acid group of oxybate and a hydroxyl group of gamma-cyclodextrin.

In yet another embodiment, PEG-oxybate-gamma cyclodextrin double cleavage prodrugs are provided which contain an ester linkage between a hydroxyl group of oxybate and a carboxylic acid group of PEG acid and; another ester linkage between a carboxylic acid groups of two oxybate single cleavage prodrugs and two hydroxyl groups of gamma-cyclodextrin.

In yet another embodiment, PEG-oxybate-gamma cyclodextrin double cleavage prodrugs are provided which contain an ester linkage between a hydroxyl group of oxybate and a carboxylic acid group of PEG acid and another ester linkage between carboxylic acid groups of three oxybate single cleavage prodrugs and three hydroxyl groups of gamma-cyclodextrin.

In certain embodiments, PEG-oxybate-gamma cyclodextrin double cleavage prodrugs are provided which contain an ester linkage between a hydroxyl group of oxybate and a carboxylic acid group of PEG acid and another ester linkage between carboxylic acid groups of four oxybate single cleavage prodrugs and four hydroxyl groups of gamma-cyclodextrin.

In yet another embodiment, PEG-oxybate-gamma cyclodextrin double cleavage prodrugs are provided which contain an ester linkage between hydroxyl group of oxybate and carboxylic acid group of PEG acid and another ester linkage between a carboxylic acid groups of five oxybate single cleavage prodrugs and five hydroxyl groups of gamma-cyclodextrin.

In yet another embodiment, PEG-oxybate-gamma cyclodextrin double cleavage prodrugs are provided which contain an ester linkage between hydroxyl group of oxybate and carboxylic acid group of PEG acid and another ester linkage between carboxylic acid groups of six oxybate single cleavage prodrugs and six hydroxyl groups of gamma-cyclodextrin.

In yet another embodiment, PEG-oxybate-gamma cyclodextrin double cleavage prodrugs are provide which contain an ester linkage between hydroxyl group of oxybate and carboxylic acid group of PEG acid and another ester linkage between carboxylic acid groups of seven oxybate single cleavage prodrugs and seven hydroxyl groups of gamma-cyclodextrin.

In yet another embodiment, PEG-oxybate-gamma cyclodextrin double cleavage prodrugs are provided which contain ester linkage between hydroxyl group of an oxybate and a carboxylic acid group of PEG acid and another ester linkage between carboxylic acid groups of eight oxybate single cleavage prodrugs and eight hydroxyl groups of gamma-cyclodextrin.

In yet another embodiment, PEG-oxybate-gamma cyclodextrin double cleavage prodrugs are provided which contain an ester linkage between hydroxyl group of oxybate and carboxylic acid group of PEG acid and; another ester linkage between carboxylic acid groups of 1 to “n” oxybate single cleavage prodrugs and 1 to “n” hydroxyl groups of polymerized beta-cyclodextrin.

In yet another embodiment, PEG-oxybate-gamma cyclodextrin double cleavage prodrugs are provided which contain ester linkage between hydroxyl group of oxybate and carboxylic acid group of PEG acid and; another ester linkage between carboxylic acid groups of 1 to “n” oxybate single cleavage prodrugs and 1 to “n” hydroxyl groups of polymerized alpha-cyclodextrin.

In yet another embodiment, PEG-oxybate-gamma cyclodextrin double cleavage prodrugs are provided which contain ester linkage between hydroxyl group of oxybate and carboxylic acid group of PEG acid and another ester linkage between carboxylic acid groups of 1 to “n” oxybate single cleavage prodrugs and 1 to “n” hydroxyl groups of polymerized gamma-cyclodextrin.

### Synthetic Routes

The PEG-oxybate-cyclodextrin esterification reactions may be performed using a variety of suitable reagents, conditions and routes. Variations on the following methods will be apparent to one of skill in the art. For examples, oxybate-PEG ester prodrugs and oxybate-PEG di ester conjugates, including prodrugs and mixtures thereof, be synthesized by: applying any of the hydroxyl group protection methods discussed below for protecting hydroxyl group of oxybate-PEG, applying any of the esterification methods discussed below for esterification reaction between carboxylic acid group of oxybate-PEG and hydroxyl group of beta-cyclodextrin, and/or applying any of the deprotection methods discussed above for deprotecting hydroxyl groups of oxybate.

FIGS. 1A-1B provide the structure of the unsubstituted backbone of a naturally occurring alpha-cyclodextrin (α-CD), beta-cyclodextrin (β-CD), or gamma-cyclodextrin (γ-CD). FIGS. 1A and 1B also provide the conformation of these natural cyclodextrins.

### Ester Prodrugs: Reaction Between Oxybate, PEG and Cyclodextrin

Using Polymerized Beta-cyclodextrin: Sodium oxybate+Polymerized beta-cyclodextrin having “n” number hydroxyl groups, wherein 1 to “n” number of different prodrug molecules wherein carboxylic acid groups of 1 to “n” oxybate molecules are esterified with 1 to “n” hydroxyl groups of given polymerized beta-cyclodextrin molecule, respectively.

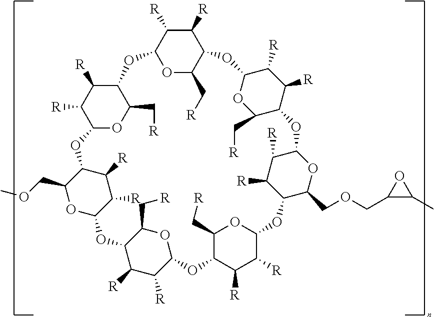

Polymerized beta-cyclodextrin is prepared by reacting β-CD with epichlorohydrin (EP) in alkaline media resulting in products that consist of a mixture of monomeric species and of polymeric fraction. The polymeric fraction of EP cross-linked β-CD with an average molecular mass up to about 20,000 appears highly water soluble.

Polymerized β-CD can be synthesized by a one-step condensation polymerization. A typical synthesis procedure is as described below: in a thermo-stated reactor vessel, a mixture of β-CD and sodium hydroxide solution are stirred for 24 h at 25° C. The mixture is then heated to 30° C. and epicholorohydrin (EP) is added rapidly, and then vigorously stirred with a magnetic stirrer. The reaction is stopped by the addition of acetone. After decantation, acetone is removed. The solution is kept at 50° C. overnight. After cooling, the solution is neutralized with HCl and ultra-filtrated in order to eliminate salt and low-molecular-weight compounds. The solution obtained is evaporated and precipitated by adding dehydrated ethanol. The product is dried under vacuum.

Beta-cyclodextrin polymerized by epichlorohydrin is also available commercially. Cylcodextrin can also be synthesized and methods of synthesis are reported in literature and are intended to be incorporated herein as references (Reference: Shufang Nie, Shu Zhang, Weisan Pan, and Yanli Liu, In vitro and in vivo studies on the complexes of glipizide with water-soluble β-cyclodextrin-epichlorohydrin polymers Drug Development and Industrial Pharmacy, 2011; 37(5): 606-612).

### PEG-Oxybate—Anion Exchange Resin Complex Prodrugs and Conjugates

In certain embodiments, oxybate-PEG double cleavage prodrugs and/or conjugates are provided which contain an ester linkage with an anion exchange resin. Such compounds may be formed by a hydroxyl group of oxybate and a carboxylic acid group of PEG acid and ionic bond between ionized carboxylic acid group of oxybate and cationic quaternary nitrogen of strong base type anion exchange resin.

An “anion exchange resin” is an insoluble organic polymer containing basic groups for exchanging anions when in a suspension with a second moiety containing anions. An example of an anion exchange resin is a cholestyramine resin, a strong base type 1 anion exchange resin powder with a polystyrene matrix and quaternary ammonium functional groups (e.g., polystyrene trimethylbenzylammonium chloride). The exchangeable anion is generally chloride which can be exchanged for, or replaced by, virtually any anionic species. A commercially available cholestyramine resins is PUROLITE™ A430MR resin. As described by its manufacturer, this resin has an average particle size range of less than 150 microns, a pH in the range of 4-6, and an exchange capacity of 1.8-2.2 eq/dry gm. Another pharmaceutical grade cholestyramine resin is available as DUOLITE™ AP143/1094 [Dow Chemical (formerly Rohm and Haas)], described by the manufacturer as having a particle size in the range of 95% less than 100 microns and 40%, less than 50 microns. The commercial literature from the suppliers of these and other resin is incorporated herein by reference (PUROLITE A-430 MR; DOW Cholestyramine USP, Form No. 177-01877-204, Dow Chemical Company; DUOLITE™ AP143/1083, Dow Chemical). In a further embodiment, DUOLITE™ AP143/1096 is selected, which has a particle size in which ≥55% of the particles are less than 150 microns and the mean diameter is ≥110 microns, as determined using dry sieving method derived from USP <811> and <796>. The Dow resins have a pH in the range of 4-6 as determined using USP <891> and an ion exchange capacity of 1.8 to 2.2 as determined using HPLC/UV of methods.

A pharmaceutically acceptable cholestyramine (USAN) (trade names Questran, Questran Light, Cholybar, Olestyr) has been approved for medical use as bile acid sequestrant. The functional group of the anion exchange resin is a quaternary ammonium group attached to an inert styrene-divinylbenzene copolymer:

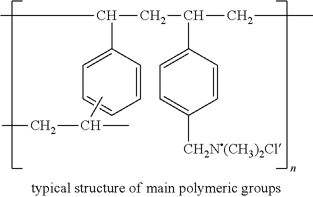

In certain embodiments, weakly basic anion exchange resins may be selected for use in the methods and/or compositions provided herein. Ion exchange resins with, secondary and tertiary amine groups as active functional groups show weak basicity, so they are called weakly basic anion exchange resins (WBAERs). There are several types of WBAERs: one with only one kind of amine groups and the other with more than two kinds of amine groups, e.g., DIAION™ WA10 (acrylic acid type with tertiary amine groups), WA20, WA21J (mixtures of and secondary amine groups), and WA30 (tertiary amine groups). For example, the weakly basic anion exchange resins include the “DIAION” ™ series [Mitsubishi Chemical Corporation], which are described by the manufacturer as having two types with different alkalinity strengths, Type I with a trimethyl ammonium group and Type II with a dimethylethanol ammonium group. Other products may comprise chitosan hydrochloride.

The oxybate—PEG-anion exchange resin complex may be prepared using a conventional ion exchange resin loading (complexing) processes or variations thereof. See, e.g., techniques described in U.S. Pat. No. 8,062,667; US Patent Publication No. US2005/181050; U.S. Pat. Nos. 8,343,546; 5,980,882 with the following modification, This complexation involves dissolving a GHIB salt (e.g., sodium oxy bate or magnesium oxybate) or an oxybate-PEG as a starting material and admixing in an aqueous medium with the water-insoluble anion exchange resin. Typically the PEG-oxybate-anion exchange resin complex thus formed is collected by filtration and washed to remove any the counterion (e.g., sodium, calcium, potassium or magnesium), unbound oxybate or by-products. Such a washing step may involve water or another aqueous solution. The complexes can be air-dried in trays, in a fluid bed dryer, or other suitable dryer, at room temperature or at elevated temperature. See, also, U.S. Pat. No. 10,398,662.

In certain embodiments, more than loading step (equilibration) is required in order to achieve the desired oxybate levels complexed onto the anion exchange resin. The use of two or more loading stages, separating the resin from the liquid phase between stages, is a means of achieving maximum loading of the oxybate onto the anion exchange resin. For example, a single equilibration (complexing) may provide about 0.1% w/w to about 20% w/w oxybate, or 1% w/w to 20% w/w, or 5% w/w to 15%, or 7% w/w to 10% w/w in the resulting oxybate-anion exchange resin complex. However, in certain embodiments, loading in excess of 25% w/w is desired, which has been found to require multiple loading steps. In certain embodiments, 25% w/w to 30% w/w oxybate, or 25% w/w to 29% w/w can be achieved, wherein the weight is based on the total weight of the PEG-oxybate-anion exchange resin complex. In certain embodiments, about 27% w/w, to 29.5% w/w oxybate is present, wherein the weight is based on the total weight of the PEG-oxybate-anion exchange resin complex. In certain embodiments, 28% w/w to 29.5% w/w oxybate is present, wherein the weight is based on the total weight of the oxybate-anion exchange resin complex. In certain embodiments, about 27% w/w oxybate is present, wherein the weight is based on the total weight of the oxybate-anion exchange resin complex. Such PEG-oxybate-anion exchange resin complexes may be admixed with suitable components to form a matrix, which PEG-oxybate-anion exchange resin complex-matrix is optionally coated.

Optionally, a non-functional seal coat may be applied to the oxybate-anion exchange resin complex, PEG-oxybate-anion exchange resin complex-matrix, and/or coated PEG-oxybate-anion exchange resin complex-optional matrix. Optionally or additionally, one or more functional coating may be provided over an PEG-oxybate-anion exchange resin complex matrix and no separate non-functional coat is utilized.

Optionally, prior to coating with a functional or non-functional coat, an PEG-oxy bate-anion exchange resin complex may be admixed with a suitable granulating agent, e.g., a hydrophilic polymer or hydrophobic excipient which forms a matrix. The resulting matrix contains the PEG-oxybate-anion exchange resin complex in a matrix with the hydrophilic or hydrophobic polymer, or hydrophobic excipient. In certain embodiments, the matrix-forming polymer or excipient is present in an amount of about 10% to about 50% by weight, of the resulting matrix, more preferably about 20% to about 40%, or about 30%, by weight. In certain of the working examples below, a “medium” molecular weight polyvinylpyrrolidone (PVP) is used. (e.g., KOLLIDON™ K30) This product, having the chemical formula (C₆H₉NO)ₙ, has a K-value of 27.0-32.4 and an average molecular weight of about 44,000 to about 54,000, in accordance with European and U.S. Pharmacopoeias. It is calculated from the relative viscosity in water [H. Flkentscher, Cellulosechemie 13 (1932): 58-64 und 71-74. See, e.g., BASF's Volker Bühler Kollidon® Polyvinylpyrrolidone excipients for the pharmaceutical industry, 9^{th} revised edition, pp. 1-330 (March 2008). For example, average molecular weight (MW) may be determined using light scattering, ultracentrifuge. Alternative methods, include number average (Mn, determined by osmometry-membrane filtration) or average viscosity (viscosity). However, other PVP, e.g. those have an average molecular weight of about 28,000 to about 100,000 which are soluble in water at room temperature may also be selected. Other suitable hydrophilic polymers and co-polymers for admixing with the complex are known, as are methods for admixture. Suitable polymers may include, e.g., propylene glycol, polyethylene glycol, polyethylene oxide, polyvinyl alcohol, mannitol, methyl cellulose, hydroxypropyl methylcellulose, hydroxypropyl cellulose, and sorbitol.

The resulting PEG-oxybate-anion exchange resin complex-matrix may contain about 5% by w eight to about 50% by weight, or about 20% to about 40% by weight, or about 25% by weight of the matrix forming polymer, with the remainder of the weight being provided by the PEG-oxybate-anion exchange resin complex.

The compositions provided herein may contain a PEG-oxybate-anion exchange resin complexes and/or other oxybate PEG ester prodrugs in immediate release form and/or in modified release form, e.g., by application of a modified release coating over the oxybate PEG ester prodrugs.

In certain embodiments, a barrier coating has a pH-independent release (i.e., it is not an enteric coating which has pH-dependent release) and is a water-insoluble, water-permeable coating material. In a preferred embodiment, neither the powder nor the liquid suspension, has an enteric or other pH-dependent coating. In certain other embodiments, a composition has a pH dependent coating over the pH-independent coating provided herein.

In one embodiment, the barrier coating layer is about is about 15% to about 80%, about 25% to about 35% by weight, about 15% w/w to about 25% by weight of the PEG-oxybate-anion exchange resin complex-optional matrix (i.e., prior to any coating) or other oxybate PEG ester prodrugs. In another embodiment, the average coating layer is about 20%, 25%, about 30%, or higher. Still other suitable ranges can be determined by one of skill in the art, having been provided with the information herein. The barrier coating is applied over the PEG-oxybate-anion exchange complex-optional matrix (e.g., as an aqueous dispersion or a solution) or other oxybate PEG ester prodrug core, dried, and milled or passed through a screen such that the barrier coated PEG-oxybate-anion exchange complex-optional matrix particles or oxybate multiparticulates are, e.g., an average of about 125 microns to about 1 mm, or an average of about 300 microns to about 900 microns, or about 400 microns to about 800 microns.

One modified release coating may be applied in the form of an aqueous dispersion containing polyvinylacetate (PVA) polymer based aqueous coating dispersion and a plasticizer. The PVA is insoluble in water at room temperature. The PVA may be used in either substantially pure form or as a blend. Suitably, a hydrophilic polymer is combined with the PVA in order to provide the PEG-oxybate ester prodrug with the desired release profile. For example, where the barrier coating comprises a PVA polymer the PVA polymer is present in an amount of about 70% to about 90% w/w of the final barrier coating layer, at least about 75%, at least about 80%, about 85% w/w of the final barrier coating layer. In one embodiment, the coating layer includes a hydrophilic polymer in an amount of about 5% to about 30% w/w, or about 10% to about 25%, or about 15% to about 20% w/w of the coating layer. The use of polyvinylpyrrolidone is described below as a suitable hydrophilic polymer. However, other suitable hydrophilic polymers may be readily selected, e.g., propylene glycol, polyethylene glycol, polyvinyl alcohol, polyvinyl pyrrolidone (e.g., KOLLIDON™ K30) mannitol, methyl cellulose, hydroxypropyl methylcellulose, hydroxypropyl cellulose, and sorbitol. Generally, a plasticizer is used in the percent range, or a mixture of plasticizers combine to total about 2 to about 50% by weight of the coating layer, more preferably about 2.5% to about 20% by weight of the coating layer on the coated PEG-oxybate ester prodrug. Preferably a plasticizer is in a range of about 2.5 to about 15% by weight of the coating layer based on the coated complex provides the most desirable properties. Suitable plasticizers may be water soluble and water insoluble. Examples of suitable plasticizers include, e.g., dibutyl sebacate, propylene glycol, polyethylene glycol, polyvinyl alcohol, triethyl citrate, acetyl triethyl citrate, acetyl tributyl citrate, tributyl citrate, triacetin, and Soluphor® P (2-pyrrolidone), and mixtures thereof. Other plasticizers are described in patent application publication US 2003/0099711 A1, May 29, 2003, page 4 (0041) the disclosure of which is incorporated herein by reference.

A commercial polyvinylacetate blend contains primarily a polyvinylacetate polymer, a stabilizer, and minor amounts of a surfactant such as sodium lauryl sulfate. Where the barrier coating comprises PVP as the stabilizer component, the final barrier coating layer generally contains about 5 to about 10% w/w of polyvinyl pyrrolidone. In one desired embodiment, the aqueous based barrier coating solution is KOLLICOAT® SR 30 D (BASF Corporation) and whose composition is about 27% PVA polymer, about 2.7% polyvinylpyrrolidone (PVP), about 0.3% sodium lauryl sulfate (solids content 30% w/w), mixed with a plasticizer. Generally, such stabilizing components are present in an amount totaling less than about 10% w/w, and preferably less than about 5% w/w. See, also, U.S. Pat. Nos. 6,066,334 and 6,026,277, which is incorporated by reference herein. Where this product is selected, additional hydrophilic polymer may be added to facilitate a more rapid release, i.e., in less than 8 hours as described herein. Optionally, a selected surfactant is present in an amount of about 1% or less. In one embodiment, the surfactant is a non-ionic surfactant. Optionally, an ionic surfactant may be selected.

In a particularly desirable embodiment, the desired modified release is obtained when the coating layer formed by application of the aqueous dispersion containing the KOLLICOAT® SR-30D-plasticizer is dried and cured. Preferably, the coating is cured for about 1 to about 24 hours. In alternate embodiments, the coating is cured for about 4 to about 16 hours, and preferably about 5 hours at high temperature, e.g., about 50° C. to about 65° C., and preferably about 60° C. See, e.g., US Published Patent Application No. US 2007/0215511A, published Sep. 20, 2007, and its counterpart application, WO 2007/109104, the disclosures of which are incorporated herein by reference.

Optionally, another barrier coating may be selected. In another embodiment, a non-aqueous solvent-based ethylcellulose, such as commercially available as the ETHOCEL™ products (Dow) or an aqueous SURELEASE® may be modified in order to achieve the barrier coating characteristics defined herein, e.g., by addition of a sufficient amount of plasticizer to improve flexibility and/or by curing to a sufficient temperature to achieve the desired release rate. Dow's web site describes three of these products, Std 7 (viscosity of 6-8 mPa-s (CP); Std 10 (9-11 mPa-s (CP)); Std 20 (18-22 mPa-s (CP)), each of which has a 48.0-49.5% ethoxyl content) as being useful for tablet coating. Further, optionally combining one of these polymers in combination with a water-soluble active and/or water-soluble excipient such as a METHOCEL™ cellulose ether and/or CARBOWAX™ polyethylene glycols is further described. Alternatively, it may be possible to modify an aqueous based ethylcellulose barrier coating in order to achieve the modified release barrier coating characteristics required herein, e.g., by addition of a sufficient amount of plasticizer to improve flexibility and/or by curing to a sufficient temperature to achieve the desired release rate. See, e.g., the barrier coatings described in U.S. Pat. Nos. 6,066,334 and 6,046,277; see, also, e.g., U.S. Pat. Nos. 6,046,277 and 6,001,392; US Published Patent Application No. 2003/0099711 and related application WO 03/020242; WO 2006/022996 and related applications US Published Patent Application Nos. US 2005/0232986; US 2005/0232987; US 2005/0232993; US 2005/0266032; U.S. Pat. Nos. 7,067,116; 6,667,058, 6,001,392, the disclosures of which are incorporated herein by reference, among others.

It may be possible utilize other aqueous or non-aqueous solvent based systems which do not require curing. For example, an aqueous based acrylic polymer (a EUDRAGIT® RL30D and EUDRAGIT® RS30D blend is described herein), but requires the addition of anti-tacking agent in order to facilitate processing and even coating. Suitable anti-tacking agents include, e.g., talc, glycerol monostearate (GMS), and mixtures thereof. These agents are present in an amount of about 0.2%-4.5% w/w based on the dry weight of the coating polymer applied to form the coating layer of the modified release component. Typically, the coating layer resulting from application of an acrylic polymer based coating does not require curing.

In one embodiment, the coating may be a EUDRAGIT® brand acrylate based coating materials [including, e.g., a poly (ethyl acrylate-co-methyl methacrylate-co-trimethylammonioethyl methacrylate chloride) polymer system]. For example, EUDRAGIT® RS 30D [a pH-independent, 30% aqueous dispersion of poly (ethyl acrylate-co-methyl methacrylate-co-trimethylammonioethyl methacrylate chloride) 1:2:0.1)], or EUDRAGIT®RL 30D [a 30% aqueous dispersion, pH independent polymer, poly (ethyl acrylate-co-methyl methacrylate-co-trimethylammonioethyl methacrylate chloride) 1:2:0.2)] may be selected as the barrier coating. In one embodiment, a blend of EUDRAGIT® RS 30D and EUDRAGIT® RL 30D may be prepared to optimize the hydrophilicity or hydrophobicity of the film in order to achieve desirable release profiles.

A coating as described herein may be applied using techniques described by the polymer manufacturer and/or techniques which are known to those of skill in the art. Suitable methods and apparatus have been described in the patent and non-patent literature and include, e.g., spraying in a fluid bed processor. The coating solution can be sprayed in a fluid bed processor (e.g., VECTOR™ FLM-1 fluid bed processor) using Wurster process.

The coated oxybate-PEG prodrug is then dried and/or cured.

### Double Cleavage Prodrug: Di Ester Prodrugs

Step I: Single Cleavage Prodrug of Oxybate

HO—CH₂—CH₂—CH₂—COO⁻Na⁺+mPEG-OCH₂—COOH→mPEG-O—CH₂—COO—CH₂—CH₂—CH₂—COO⁻Na⁺+H₂O

Step II

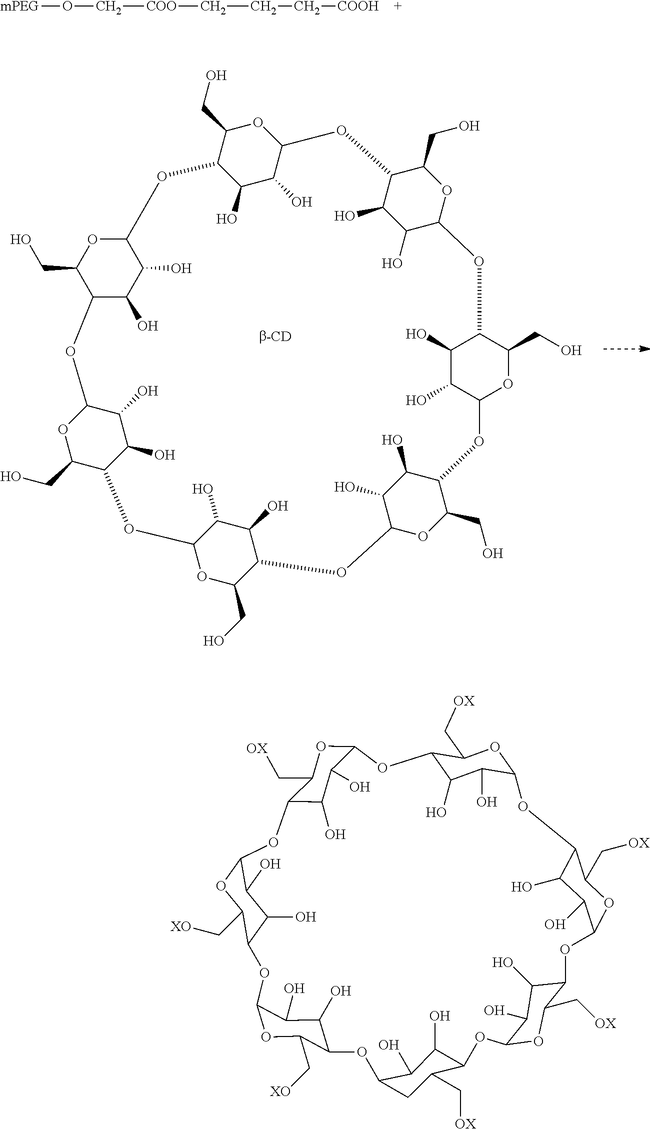

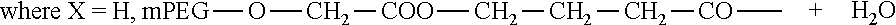

The following illustrates synthetic methods for generating the oxybate-PEG ester compounds described herein. These methods are illustrative only and not a limitation on the invention.

### Ester Prodrugs (Single Cleavage Type): Reaction Between Oxybate Salt and PEG Acid

HO—CH₂—CH₂—CH₂—COO⁻Na⁺mPEG-OCH₂—COOH→mPEG-O—CH₂—COO—CH₂—CH₂—CH₂—COO⁻Na⁺+H₂O Single cleavage prodrug of oxybate

### Double Cleavage Prodrug: Anion Exchange and Ester Hydrolysis as Cleavage Steps

Step I

HO—CH₂—CH₂—CH₂—COO⁻Na⁺+mPEG-OCH₂—COOH→mPEG-O—CH₂—COO—CH₂—CH₂—CH₂—COO⁻Na⁺H₂O Single cleavage prodrug of oxybate

Step II: Single Cleavage Prodrug

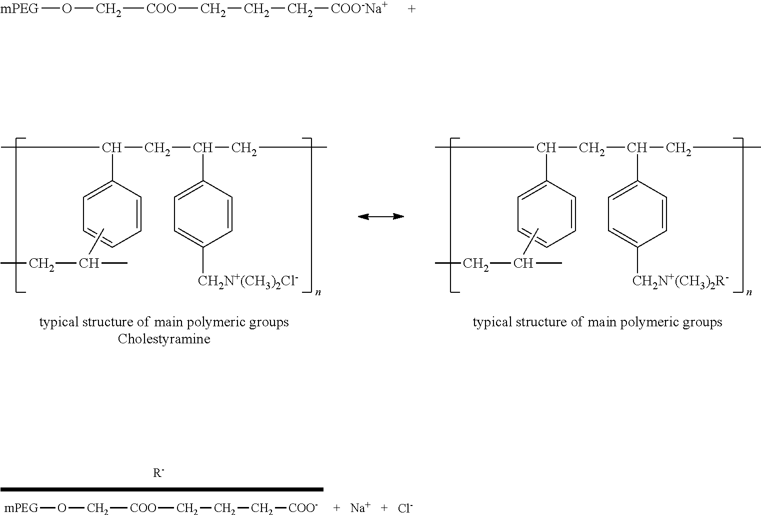

### Double Cleavage Prodrug

Alternatively, an oxybate cholestyramine anion exchange complex can be prepared first and then free hydroxyl group of oxybate in the ion exchange complex can be esterified with carboxylic acid group of methoxy PEG acid (mPEG acid). See, e.g., U.S. Pat. Nos. 10,398,662 and 8,062,667, for techniques suitable for forming drug-ion exchange complexes.

### Double Cleavage Prodrug: Di Ester Prodrugs

Step I: Single Cleavage Prodrug of Oxybate

HO—CH₂—CH₂—CH₂—COO⁻Na⁺+mPEG-OCH₂—COOH→mPEG-O—CH₂—COO—CH₂—CH₂—CH₂—COO⁻Na⁻+H₂O

Step II

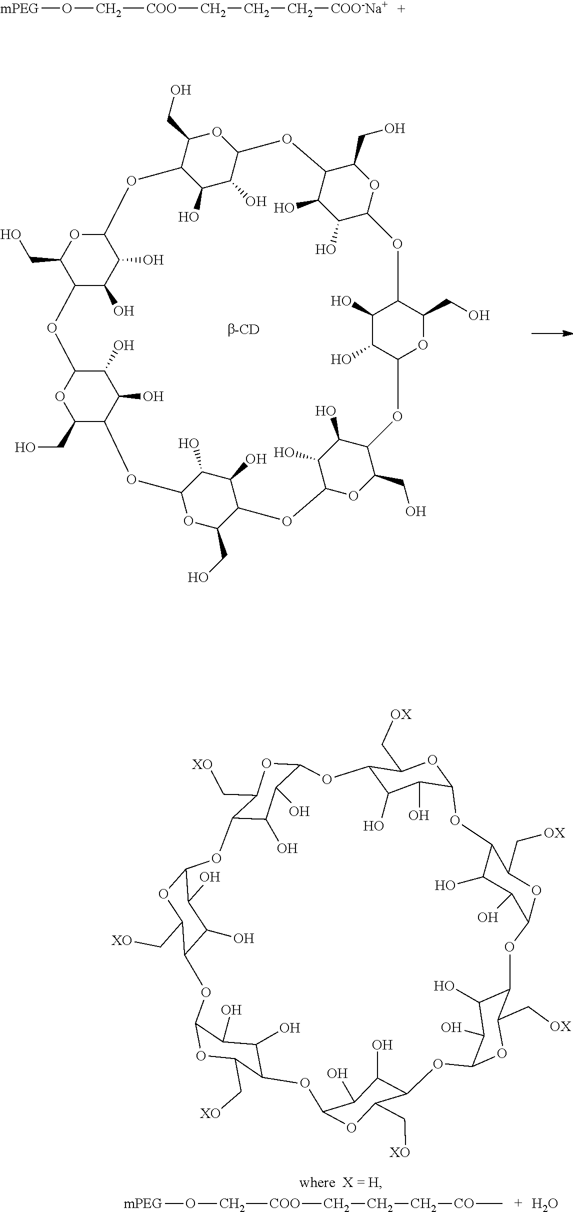

Alternatively, oxybate-betacyclodextrin ester prodrug can be prepared first and then free hydroxyl group of oxybate in such prodrug can be further esterified with carboxylic acid group of mPEG acid to obtain double cleavage prodrugs.

Similarly, di ester prodrugs containing ester linkage between OH group of oxybate and COOH of mPEG acid and another ester linkage between COOH group of oxybate and OH group of alpha-cyclodextrin can be synthesized.

Similarly, di ester prodrugs containing ester linkage between OH group of oxybate and COOH of mPEG acid and another ester linkage between COOH group of oxybate and OH group of gamma-cyclodextrin can be synthesized.

Formation of Anion Exchange Resin Complexation Based Double Cleavage Prodrugs

Step 1: Single Cleavage Prodrug of Oxybate

HO—CH₂—CH₂—CH₂—COO⁻Na⁺+mPEG-OCH₂—COOH→mPEG-O—CH₂—COO—CH₂—CH₂—CH₂—COO⁻Na⁺+H₂O

Step II: Oxybate-PEG Single Cleavage Prodrug can be Treated with Cholestyramine to Form Double Cleavage Prodrugs.

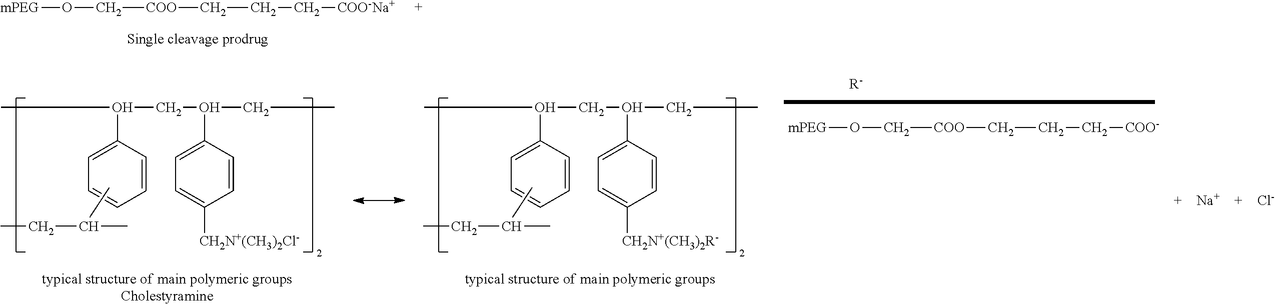

Double Cleavage Prodrug

Single cleavage oxybate-PEG ester prodrug is dissolved in deionized water. Cholestyramine is suspended in the same solution under stirring. Stirring is continued for 3 hours to form anion exchange complex between cholestyramine and single cleavage oxybate-PEG ester prodrug. Ion exchange complexation is equilibrium reaction. The complex is separated, dried and sized. Formed complex is again suspended in single cleavage oxybate-PEG ester prodrug solution for another equilibrium. The multi stage equilibrium is performed 5 times to ensure maximum complexation efficiency.

In certain embodiments, a composition may comprise one or more different oxybate-PEG ester compounds, including, e.g., one or more different PEGylated oxybate-anion exchange resin complexes, optionally, in a matrix, and optionally coated.

In certain embodiments, a composition may comprise one or more oxybate-PEG ester compounds and one or more different non-PEGylated oxybate-anion exchange resin complexes, optionally, in a matrix, and optionally coated.

### Esterification Reactions

Esterification is the general name for a chemical reaction in which two reactants (typically an alcohol and an acid) form an ester as the reaction product. Esterification reactions typically involve reaction between carboxylic acid (—COOH) group of one reactant and hydroxyl (—OH) group of second reactant. Esters are derived from substitution reaction of a carboxylic acid and an alcohol.

Esterification of carboxylic acids with alcohols: The classic synthesis is the Fischer esterification, which involves treating a carboxylic acid with an alcohol in the presence of a dehydrating agent: RCO₂H+R′OH⇄RCO₂R′+H₂O

The equilibrium constant for such reactions is about 5 for typical esters, e.g., ethyl acetate. [Williams, Roger J.; Gabriel, Alton; Andrews, Roy C. (1928). “The Relation Between the Hydrolysis Equilibrium Constant of Esters and the Strengths of the Corresponding Acids”. J. Am. Chem. Soc. 50 (5): 1267-1271. doi:10.1021/ja01392a005.] The reaction is slow in the absence of a catalyst. Sulfuric acid is a typical catalyst for this reaction. Many other acids are also used such as polymeric sulfonic acids.

Since esterification is highly reversible, the yield of the ester can be improved using Le Chatelier's principle: Using the alcohol in large excess (i.e., as a solvent). Using a dehydrating agent: sulfuric acid not only catalyzes the reaction but sequesters water (a reaction product). Other drying agents such as molecular sieves are also effective. Removal of water by physical means such as distillation as a low-boiling azeotropes with toluene, in conjunction with a Dean-Stark apparatus.

Reagents are known that drive the dehydration of mixtures of alcohols and carboxylic acids. One example is the Steglich esterification, which is a method of forming esters under mild conditions. The method is popular in peptide synthesis, where the substrates are sensitive to harsh conditions like high heat. DCC (dicyclohexylcarbodiimide) is used to activate the carboxylic acid to further reaction. 4-Dimethylaminopyridine (DMAP) is used as an acyl-transfer catalyst. [B. Neises; W. Steglich. “Esterification of Carboxylic Acids with Dicyclohexylcarbodiimide/4-Dimethylaminopyridine: tert-Butyl ethyl fumarate”. Organic Syntheses.; Collective Volume, 7, p. 93]

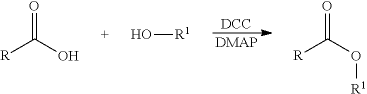

Another method for the dehydration of mixtures of alcohols and carboxylic acids is the Mitsunobu reaction: RCO₂H+R′OH+P(C₆H₅)₃+R₂N₂→RCO₂R′+OP(C₆H₅)₃+R₂N₂H₂

Carboxylic acids can be esterified using diazomethane: RCO₂H+CH₂N₂→RCO₂CH₃+N₂. Using this diazomethane, mixtures of carboxylic acids can be converted to their methyl esters in near quantitative yields, e.g., for analysis by gas chromatography. The method is useful in specialized organic synthetic operations but is considered too hazardous and expensive for large-scale applications.

Esterification of Carboxylic Acids with Epoxides

Carboxylic acids are esterified by treatment with epoxides, giving β-hydroxyesters: RCO₂H+RCHCH₂O→RCO₂CH₂CH(OH)R. This reaction is employed in the production of vinyl ester resin resins from acrylic acid.

Alcoholysis of acyl chlorides and acid anhydrides. Alcohols react with acyl chlorides and acid anhydrides to give esters:

RCOCl+R′OH→RCO₂R′+HCl

(RCO)₂O+R′OH→RCO₂R′+RCO₂H

The reactions are irreversible simplifying work-up. Since acyl chlorides and acid anhydrides also react with water, anhydrous conditions are preferred. The analogous acylations of amines to give amides are less sensitive because amines are stronger nucleophiles and react more rapidly than does water. This method is employed only for laboratory-scale procedures, as it is expensive.

Alkylation of Carboxylate Salts

Although not widely employed for esterifications, salts of carboxylate anions can be alkylating agent with alkyl halides to give esters. [Riemenschneider, Wilhelm; Bolt, Hermann M. “Esters, Organic”. Ullmann's Encyclopedia of Industrial Chemistry. Weinheim: Wiley-VCH. doi:10.1002/14356007.a09_565] In the case that an alkylchloride is used, an iodide salt can catalyze the reaction (Finkelstein reaction). The carboxylate salt is often generated in situ. [Matsumoto, Kouichi; Shimazaki, Hayato; Miyamoto, Yu; Shimada, Kazuaki; Haga, Fumi; Yamada, Yuki; Miyazawa, Hirotsugu; Nishiwaki, Keiji; Kashimura, Shigenori (2014). “Simple and Convenient Synthesis of Esters from Carboxylic Acids and alkyl Halides Using Tetrabutylammonium Fluoride”. Journal of Oleo Science. 63 (5): 539-544.] In difficult cases, the silver carboxylate may be used, since the silver ion coordinates to the halide aiding its departure and improving the reaction rate. This reaction can suffer from anion availability problems and, therefore, can benefit from the addition of phase transfer catalysts or highly polar aprotic solvents such as DMF.

Transesterification: Transesterification, which involves changing one ester into another one, is widely practiced: RCO₂R′+CH₃OH→RCO₂CH₃+R′OH

Like the hydrolysation, transesterification is catalysed by acids and bases. The reaction is widely used for degrading triglycerides, e.g. in the production of fatty acid esters and alcohols. Poly(ethylene terephthalate) is produced by the transesterification of dimethyl terephthalate and ethylene glycol: [Riemenschneider, Wilhelm; Bolt, Hermann M. “Esters, Organic”. Ullmann's Encyclopedia of Industrial Chemistry. Weinheim: Wiley-VCH. doi:10.1002/14356007.a09_565]

(C₆H₄)(CO₂CH₃)₂+2C₂H₄(OH)₂→l/n{(C₆H₄)(CO₂)₂(C₂H₄)}n+2CH₃OH

A subset of transesterification is the alcoholysis of diketene. This reaction affords 2-ketoesters [Riemenschneider, Wilhelm; Bolt, Hermann M. “Esters, Organic”. Ullmann's Encyclopedia of Industrial Chemistry. Weinheim: Wiley-VCH. doi:10.1002/14356007.a09_565]. (CH₂CO)₂+ROH→CH₃C(O)CH₂CO₂R

Carbonylation

Alkenes undergo “hydro-esterification” in the presence of metal carbonyl catalysts.

Esters of propionic acid are produced commercially by this method:

C₂H₄+ROH+CO→C₂H₅CO₂R

A preparation of methyl propionate is one illustrative example.

C₂H₄+CO+MeOH→MeO₂CCH₂CH₃

The carbonylation of methanol yields methyl formate, which is the main commercial source of formic acid. The reaction is catalyzed by sodium methoxide:

CH₃OH+CO→CH₃O₂CH

Addition of carboxylic acids to alkenes and alkynes

In the presence of palladium-based catalysts, ethylene, acetic acid, and oxygen react to give vinyl acetate:

C₂H₄+CH₃CO₂H+½O₂→C₂H₃O₂CCH₃+H₂O

Direct routes to this same ester are not possible because vinyl alcohol is unstable. Carboxylic acids also add across alkynes to give the same products.
Silicotungstic acid is used to manufacture ethyl acetate by the alkylation of acetic acid by ethylene:

C₂H₄+CH₃CO₂H→CH₃CO₂C₂H₅

### Protection of Hydroxyl Compounds as Benzyl Ether and Deprotection

Protection

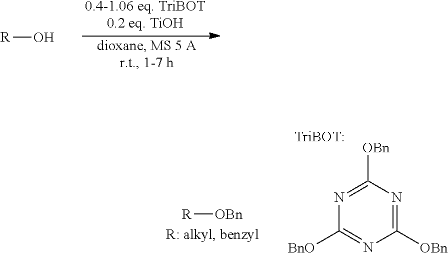

Inexpensive stable crystalline 2,4,6-tris(benzyloxy)-1,3,5-triazine (TriBOT) can be used as an acid-catalyzed O-benzylating reagent. The reaction of various functionalized alcohols with 0.4 equiv of TriBOT in the presence of trifluoromethanesulfonic acid afford benzyl ethers. TriBOT, which is the formal trimerization of the smallest unit of benzyl imidate, offers high atom economy. [See, e.g., K. Yamada, H. Fujita, M. Kunishima, Org. Lett., 2012, 14, 5026-5029.]

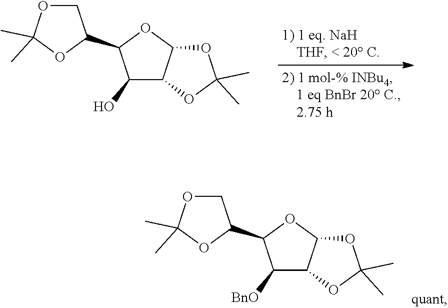

A fast, quantitative benzylation of hindered sugar hydroxyls with NaH/THF is possible in the presence of a catalytic amount of the quaternary ammonium salt IN(Bu)₄. A sample procedure with catalyst produces quantitative yield after 10 minutes (min) to 165 min at room temperature versus 24 h at reflux with excess benzyl bromide and no catalyst. [See, e.g., Czernecki, et al, New Method for the Benzylation of Hindered Sugar Hydroxyls Tetrahedron Lett., 1976, 17, 3535-3536.]

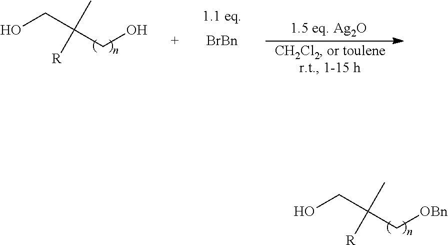

Treatment of a symmetrical diol with Ag₂O and an alkyl halide give monoprotected derivative. [See, e.g., A. Bouzide, G. Sauvé, Highly selective silver(I) oxide mediated mono-protection of symmetrical diols Tetrahedron Lett., 1997, 38, 5945-5948.]

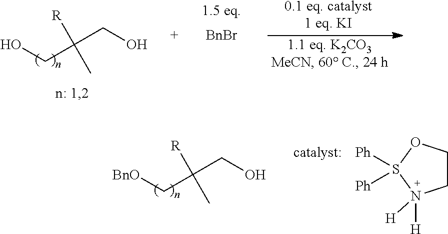

Diarylborinic acid catalysis is an efficient and general method for selective acylation, sulfonylation, and alkylation of 1,2- and 1,3-diols. The efficiency, generality, and operational simplicity of this method are competitive with those of the broadly applied organotin-catalyzed reactions. A mechanism is suggested, in which a tetracoordinate borinate complex reacts with the electrophilic species in the turnover-limiting step of the catalytic cycle. [Reference: D. Lee, C. L. Williamson, L. Chan, M. S. Taylor Regioselective, Borinic Acid-Catalyzed Monoacylation, Sulfonylation and alkylation of Diols and Carbohydrates: Expansion of Substrate Scope and Mechanistic Studies J. Am. Chem. Soc., 2012, 134, 8260-8267.]

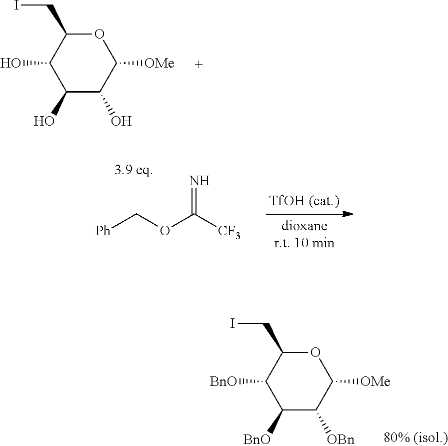 [SMILES: CO[C@H]1O[C@H](CI)[C@@H](O)[C@H](O)[C@H]1O ; N=C(OCc1ccccc1)C(F)(F)F ; CO[C@H]1O[C@H](CI)[C@@H](OCc2ccccc2)[C@H](OCc2ccccc2)[C@H]1OCc1ccccc1]

Two methods are described for the regioselective displacement of the hydroxy group in methyl glycosides with iodide. Products of the first method employing triphenylphosphine and iodine need purification on a reverse phase column. A one-pot procedure via sulfonates and subsequent substitution with iodide and methods for the protection of the iodoglycosides are also described. See, e.g., P. R. Skaanderup, C. S. Poulsen, L. Hyldtoft, M. R. Jorgensen, R. Madsen, Synthesis, 2002, 1721-1727].

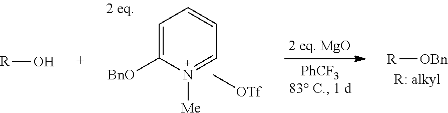

2-Benzyloxy-1-methylpyridinium triflate is a stable, neutral organic salt that converts alcohols into benzyl ethers upon warming. Benzylation of a wide range of alcohols occurs in very good yield. [See, e.g., K. W. C. Poon, G. B. Dudley Mix-and-Heat Benzylation of Alcohols Using a Bench-Stable Pyridinium Salt, J. Org. Chem., 2006, 71, 3923-3927.]

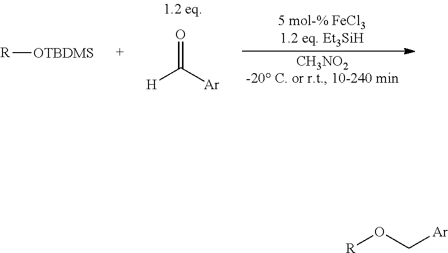

Various silyl ethers are readily and efficiently transformed into the corresponding alkyl ethers in high yields by the use of aldehydes combined with triethylsilane in the presence of a catalytic amount of iron(III) chloride. [See, e.g., K. Iwanami, K. Yano, T. Oriyama, Synthesis, 2005, 2669-2672.]

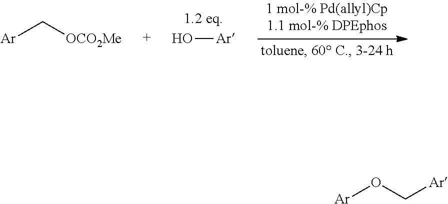

A catalytic amount of Pd(η³-C₃H₅)Cp and DPEphos as ligand are efficiently converted aryl benzyl carbonates into benzyl-protected phenols through a decarboxylative etherification. Alternatively, the nucleophilic substitution of benzyl methyl carbonates with phenols proceeded in the presence of the catalyst, yielding aryl benzyl ethers. [Reference: R. Kuwano, H. Kusano, Org. Lett., 2008, 10, 1795-1798.]

Deprotection

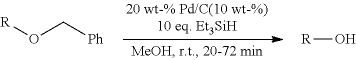

In-situ generation of molecular hydrogen by addition of triethylsilane to palladium on charcoal results in rapid and efficient reduction of multiple bonds, azides, imines, and nitro groups, as well as deprotection of benzyl and allyl groups under mild, neutral conditions. [See, e.g., P. K. Mandal, J. S. McMurray, J. Org. Chem., 2007, 72, 6599-6601]

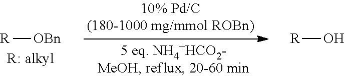

Transfer hydrogenation utilizing palladium on carbon and formic acid provides a fast and simple removal of O-benzyl groups from carbohydrate derivatives. However, when formic acid is the hydrogen donor, a large amount of palladium has to be used. [Reference: T. Bieg, W. Szeja, Synthesis, 1985, 76-77]

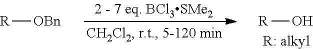

An efficient and convenient method allows the removal of benzyl ether protecting groups in the presence of other functionality. Varying the solvent allows the removal of trityl groups in the presence of benzyl ethers. [See, e.g., M. S. Congreve, E. C. Davison, M. A. M. Fuhry, A. B. Holmes, A. N. Payne, R. A. Robinson, S. E. Ward Selective cleavage of benzyl ethers [Letter], Synlett, 1993, 663-664.]

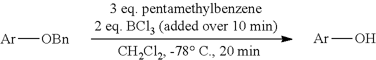

A chemoselective debenzylation of aryl benzyl ethers proceeds at low temperature with a combination of BCl₃ and pentamethylbenzene as a cation scavenger in the presence of various functional groups. [See, e.g., Reference: K. Okano, K.-i. Okuyama, T. Fukuyama, H. Tokuyama, Synlett, 2008, 1977-1980.]

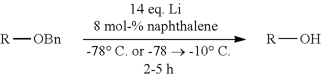

The reaction of different protected alcohols, amines and amides with lithium and a catalytic amount of naphthalene in THF at low temperature leads to their deprotection under very mild reaction conditions, the process being in many cases chemo-selective. [See, e.g., E. Alonso, D. J. Ramón, M. Yus, Reductive deprotection of allyl, benzyl and sulfonyl substituted alcohols, amines and amides using a naphthalene-catalysed lithiation Tetrahedron, 1997, 53, 14355-14368.]

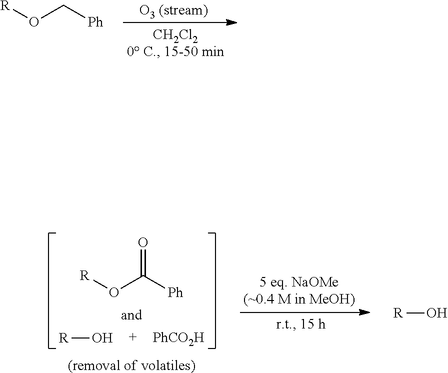

Benzyl ether protective groups are oxidatively removed by ozone under relatively mild conditions. Reaction products are benzoic ester, benzoic acid, and the corresponding alcohol. Subsequent deacylation with sodium methoxide affords a convenient debenzylation technique which has been applied to various O-benzyl protected carbohydrates. [See, e.g., P. Angibeaud, J. Defaye, A. Gadelle, J.-P. Utille, Synthesis, 1985, 1123-1125]

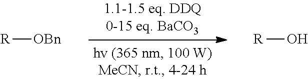

The deprotection of benzyl ethers is effectively realized in the presence of 2,3-dichloro-5,6-dicyano-p-benzoquinone (DDQ) in MeCN under photoirradiation using a long wavelength UV light. [See, e.g, M. A. Rahim, S. Matsumura, K. Toshima, Tetrahedron Lett., 2005, 46, 7307-7309].

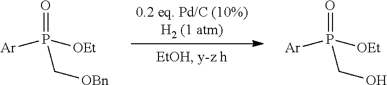

Arylhydroxymethylphosphinic acid derivatives are prepared by a palladium(0) catalysed arylation of ethyl benzyloxymethylphosphinate with aryl halides followed by subsequent hydrogenolysis of the benzyl protecting group and hydrolysis of the ester function. [See, e.g., H.-J. Cristau, A. Hervé, F. Loiseau, D. Virieux, Synthesis, 2003, 2216-2220]

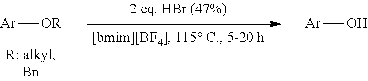

The ionic liquid [bmim][Br] confers high nucleophilicity on the bromide ion for the nucleophilic displacement of an alkyl group to regenerate a phenol from the corresponding aryl alkyl ether in the presence of p-toluenesulfonic acid. Dealkylation of various aryl alkyl ethers could also be achieved using stoichiometric amounts of concentrated hydrobromic acid in [bmim][BF₄]. [See, e.g., S. K. Boovanahalli, D. W. Kim, D. Y. Chi, J. Org. Chem., 2004, 69, 3340-3344]

In certain embodiments, the final formulation contains, at a minimum, at least one oxybate-PEG ester which may be in immediate release form and/or may have one or more of a moisture barrier coating, a non-functional aesthetic coating, a pH-independent modified release barrier coating, or a pH-dependent enteric coating optionally with one or more of a moisture barrier coating, a non-functional aesthetic coating, a pH-independent modified release barrier coating, or a pH-dependent enteric coating to afford a coated particulate oxybate-PEG ester powder which can be used to generate a solid dosage form or a liquid dosage form.

By “immediate release” is intended a composition that releases >95% of the oxybate cyclodextrin drug(s) in immediate release form substantially completely into the gastrointestinal tract of the user within a period of less than an hour, usually between about 0.1 and about 1 hour and less than about 0.75 hours from ingestion. For example, in certain embodiments, immediate release may be greater than 80% drug release in 0.1NHCl in one hour.

As used herein, the term “modified release” includes sustained release, controlled release, prolonged release, delayed release, timed release, retarded release, slow release, and extended release. The release rate can be controlled by the use of modified release polymers such those described herein.

As used herein “delayed release” refers to a formulation (or a coating) which does releases the active component into the gastrointestinal tract after a delay or lag period, e.g., after one hour, two hours, or longer.

A composition as provided herein may have oxybate in more than one form within a single composition. In such an instance, the final product may be termed an extended release composition, which may contain both a modified release component and an immediate release composition. In certain embodiments, a delayed release composition has no immediate release component.

In certain embodiments, one or more of the oxybate cyclodextrin ester compounds is in both immediate release and extended release forms.

As used herein, a “non-functional coating” refers to a coating which contributes no detectable modified drug release functions to a coated drug, e.g., and may alternatively be referred to as a “seal coat”. In other words, such a coating will not change the drug release profile of an immediate release drug to a modified release profile. The non-functional coating may contain a polymer, or a non-polymeric material, which is a moisture barrier to preserve the integrity of the coated drug (granules) during storage and/or further processing. The non-functional coating may additionally or alternatively comprise oxygen barrier properties. In certain embodiments, the non-functional coating assists in protecting the coated drug product against interaction with the packaging in which the product is stored (e.g., an aluminium foil pouch). As provided herein, some of these non-functional materials are available commercially. See, e.g., a low molecular hypromellose, methylhydroxy ethylcellulose, hydroxypropyl cellulose, polyvinyl pyrrolidone, polyvinyl alcohol (see, e.g., Opadry® II (polyvinyl alcohol, polyethylene glycol, talc) or Opadry® AMB (Colorcon Inc., Harleysville, Pa.), sodium carboxymethyl cellulose; a combination of polyacrylic resin, carbomer, and a powdered cellulose coat. As used herein, a “low molecular weight” has a viscosity of about 100 cP to about 5000 cP. Weight percentages of these non-functional coatings, where present, are provided as weight added, in an amount of about 1% to about 50%, or about 10% to about 40%, or about 20% to about 30% weight added to the finished particle.

In certain embodiments, the final formulation is a powder for suspension which, following oral ingestion by a patient (human subject), has a therapeutic effect over at least about 3.5 hours to about 8 hours. The formulations provided herein may be packaged, stored, and shipped as a powder designed for reconstitution in an aqueous liquid (e.g., drinking water) prior to dosing as a liquid suspension. In other embodiments, two or more different types of oxybate-PEG esters formulated together into a solid dose tablet, or loaded into capsules.

As an added safety feature, the composition may further contain a pharmaceutically acceptable dye which dissolves if the composition is placed in an aqueous liquid to provide a distinctive color.

1. Powder Formulation
To provide a final powder formulation suitable for reconstitution in water, the powder oxybate PEG ester(s) may be mixed with optional excipients, flow agents such as a glidant or lubricant, preservatives, pH buffering agent, viscosity building agent or the like may be admixed and the powder packaged into a suitable pack (e.g., aluminium foil sachet) or bottle. In certain embodiments, the powder includes buffering agents designed to adjust the pH to the range of about 7 to about 8, or about 7.2 to 7.8, or about 7.5.
Optionally, one or more desired excipients, including, e.g., flavorants, sweeteners, viscosity builders, flow-aids, pH-adjuster, preservatives, or other excipients may be blended into the final powder mix and/or added to the suspension.

2. Tablet Formulations

The oxybate-PEG tablets may be prepared using an oxybate-PEG ester, and one or more of a filler, one or more disintegrant, one or more binder, one or more a buffering agent, one or more lubricant, one or more glidant, or blends of these components. Suitably, the tablets also include taste and/or mouth feel enhancers including, e.g., one or more of a sweetener, a flavorant, a gum, or blends of these components.

In one embodiment, a tablet is formulated as an orally disintegrating tablet. Such orally disintegrating tablets may disintegrate in the mouth in less than about 30 seconds or less. In another embodiment, a tablet is formulated as a chewable tablet.

Typically, a chewable tablet will contain a filler or a mixture of fillers in the range of about 10% w/w to about 90% w/w, about 50% w/w to about 85% w/w, or about 50% w/w to about 70% w/w of the total tablet weight. Suitable fillers may include, e.g., mannitol, lactose, maltose, fructose, sucrose, xylitol, maltitol, microcrystalline cellulose, dicalcium phosphate, guar gum, xanthan gum, tragacanth gum, pre-gelatinized starch, compressible sugar, calcium carbonate, magnesium carbonate, calcium sulfate, dextrates, maltodextrin. In one embodiment, a chewable tablet contains a blend of mannitol, xanthan gum, microcrystalline cellulose, and guar gum in an amount of about 60% w/w to about 75% w/w. In one embodiment, a gum or a combination of gums is provided in an amount of about 0.25% w/w to about 5% w/w, or about 0.25% to about 1% w/w. In another embodiment, microcrystalline cellulose is provided in an amount of about 5% w/w to about 25% w/w, or about 10% w/w to about 15% w/w based on the total tablet weight prior to any non-functional coating. A product containing a combination of microcrystalline cellulose and guar gum is commercially available as Avicel®, which contains a ratio of 80 parts by weight microcrystalline cellulose to 20 parts by weight guar gum. This blend of microcrystalline cellulose (MCC) and guar gum may be present in an amount of about 5% w/w to about 25% w/w of the total tablet weight.

A chewable tablet as described herein will also contain a disintegrant or blend of disintegrants in the range of about 1% w/w to about 25% w/w, or about 5% w/w to about 15% w/w, or about 10% w/w to about 14% w/w based on the total tablet weight. Suitable disintegrants include, e.g., crospovidone, sodium starch glycollate, croscarmellose sodium, carboxymethyl cellulose sodium, carboxymethylcellulose calcium, starch. In one embodiment, a tablet as described herein contains crospovidone in a range of about 5% w/w to about 10% w/w, or about 12% w/w based on the tablet weight prior to any non-functional coating being applied.

The binder for the chewable tablet may be absent (i.e., 0%), or optionally, present in an amount of about 1% w/w to about 15% w/w of the total tablet weight. Examples of suitable binders include polyvinylpyrrolidone (Povidone), hydroxypropyl methyl cellulose, hydroxypropyl cellulose, hydroxyethyl cellulose, methyl cellulose, polyvinyl alcohol, starch, acacia, alginic acid, sodium alginate.

In one embodiment, the chewable tablet contains a sweetener in an amount of about 0.01% w/w to about 3% w/w, or about 0.5% w/w to about 2% w/w, or about 1% w/w to about 2% w/w or about 1.5% w/w, based on the total tablet weight exclusive of any optional non-functional coating. Suitable sweeteners may include, e.g., Aspartame, Saccharin, Saccharin sodium, Sucralose, Sodium cyclamate, Xylitol, Acesulfamate Potassium, and blends thereof. Optionally, in addition to functioning as a sweetener, an excipient may function as a filler. Examples of suitable sweeteners/fillers including, e.g., fructose, sucrose, xylitol, maltitol. Optionally, when performing both functions, the excipient may be present in an amount in excess of about 10% w/w of the tablet. In such an instance, additional sweetener may be omitted (e.g., present in 0% added sweetener). Alternatively, a second sweetener or a combination of sweeteners which differs from the filler is added in the amount provided in this paragraph in order to further enhance taste.

Suitably, the tablet is provided with a buffering agent in an amount of about 0.1% w/w to about 5% w/w, or about 0.5% w/w to about 1.5% w/w based on the total tablet weight. Examples of suitable buffering agents include, e.g., citric acid, tartaric acid, malic acid, lactic acid, and acceptable salts thereof, and mixtures thereof.

When an additional flavoring agent is added, the flavoring agent(s) may be added in an amount of about 0.05% w/w to about 3% w/w, or about 0.1% to about 1% w/w or about 0.5% w/w, based on the total weight of the tablet (exclusive of any optional non-functional coating). Suitable flavoring agents may include, both natural and artificial flavoring agents such as are generally available through several custom manufacturers around the world such as Fona [Illinois, US], Givaudan (Vernier, Switzerland), Ungerer & Company (Lincoln Park, N.J.), and International Flavors & Fragrances (New York, N.Y.) to name a few. Those skilled in the art will recognize that there are several commercial sources available including custom blenders. The flavorings may be blended prior to addition to the pharmaceutical composition or added separately. Still other flavoring agents such as bubble gum, cherry, strawberry, vanilla, grape, banana and other flavors or mixtures thereof may be selected.

Optionally, a colorant may be provided to the tablet to provide a desired visual appeal or trade dress. Such colorants may be added in the range of about 0.001 to about 1% w/w, or about 0.01% w/w to about 0.08% w/w or about 0.05% w/w, based on the total weight of the tablet (exclusive of any non-functional coating). Such colorants are available from a variety of sources including, e.g., Colorcon, Noveon, and Spectra. In one embodiment, no colorants are used in the tablet.

In order to facilitate production of the chewable tablet, excipients such as lubricants and glidants may be utilized. A lubricant may be utilized in an amount of about 0.1% w/w to about 5% w/w, about 0.2% w/w to about 4.5% w/w, or about 1.5% w/w to about 3% w/w of the total weight of the tablet. Examples of lubricants may include, e.g., magnesium stearate, sodium stearyl fumarate, stearic acid, zinc stearate, calcium stearate, magnesium trisilicate, polyethylene glycol, and blends thereof. In one embodiment, a glidant may be used in an amount of about 0.01% w/w to about 0.5% w/w, or about 0.1% w/w to about 0.3% w/w, based on the total weight of the tablet. Examples of suitable glidants include, e.g., silicon dioxide and tribasic calcium phosphate. In one embodiment, the glidant is silicon dioxide which is used in an amount of about 0.001% w/w to about 0.3% w/w or about 0.2% w/w.

Optionally, other excipients may be selected from conventional pharmaceutically acceptable carriers or excipients and well established techniques. Without being limited thereto, such conventional carriers or excipients include diluents, binders and adhesives (i.e., cellulose derivatives and acrylic derivatives), lubricants (i.e., magnesium or calcium stearate, or vegetable oils, polyethylene glycols, talc, sodium lauryl sulfate, polyoxy ethylene monostearate), thickeners, solubilizers, humectants, disintegrants, colorants, flavorings, stabilizing agents, sweeteners, and miscellaneous materials such as buffers and adsorbents in order to prepare a particular pharmaceutical composition. The stabilizing agents may include preservatives and anti-oxidants, amongst other components which will be readily apparent to one of ordinary skill in the art.

Still other suitable excipients may be selected.

### Uses and Therapeutic Methods

Provided herein are therapeutic methods to treat conditions amenable to treatment by sodium oxybate, such as those discussed hereinbelow, by administering an effective amount of one or more dosage forms of the invention.

The present dosage forms can be administered to treat a human afflicted with narcolepsy to reduce cataplexy and/or daytime sleepiness.

The present dosage forms can be administered to humans, particularly in the elderly (>50 years old), to improve the quality of sleep, or in conditions in which an increase in growth hormone levels in vivo is desired.

The compositions can also be used to treat fibromyalgia or chronic fatigue syndrome, e.g., to alleviate at least one symptom of fibromyalgia or chronic fatigue syndrome. See, U.S. Pat. No. 5,990,162.

The dosage forms described herein may be provided as a kit comprising, separately packaged, a container comprising an effective amount of the sodium oxybate powder composition in a sachet or other suitable package. For example, the powder may be packaged aluminium foil envelopes, or in a blister pack. The powder can be packaged in many conformations with or without desiccant or other materials to prevent ingress of water. Instruction materials or means, such as printed labeling, can also be included for their administration, e.g., sequentially over a preselected time period and/or at preselected intervals, to yield the desired levels of sodium oxybate in vivo for preselected periods of time, to treat a preselected condition.

A kit for treating a patient with a composition comprising an oxybate ester, said kit comprising (a) a container comprising a composition of claim 1; (b) a syringe; (c) a measuring cup; (d) a press-in-bottle adapter; optionally at least one empty pharmacy container with a child-resistant cap.

A daily dose of about the equivalent of about 1 mg/kg to about 50 mg/kg equivalent to sodium oxybate can be administered to accomplish the therapeutic results disclosed herein. For example, a daily dosage of about 0.5-20 g equivalent to sodium oxybate equivalent thereto can be administered, preferably about 1 to 15 g, in single or divided doses. In other embodiments, doses may range from about 1.5 g to about 9 g per night, or about 4.5 g to about 6 g per night.

The compositions described herein may be useful in the treatment of a variety of conditions amenable to treatment by sodium oxybate, such as narcolepsy to reduce cataplexy and/or daytime sleepiness, to improve the quality of sleep, or in conditions in which an increase in growth hormone levels in vivo is desired, and to treat fibromyalgia or chronic fatigue syndrome. The present dosage forms may be used to treat a host of other indications including drug and alcohol abuse, anxiety, cerebrovascular diseases, central nervous system disorders, neurological disorders including Parkinson's Disease and Alzheimer Disease, Multiple Sclerosis, autism, depression, inflammatory disorders, including those of the bowel, such as irritable bowel disorder, regional ileitis and ulcerative colitis, autoimmune inflammatory disorders, certain endocrine disturbances and diabetes.

The compositions may also be administered for the purpose of tissue protection including protection following hypoxia/anoxia such as in stroke, organ transplantation, organ preservation, myocardial infarction or ischemia, reperfusion injury, protection following chemotherapy, radiation, progeria, or an increased level of intracranial pressure, e.g. due to head trauma. The present dosage forms can also be used to treat other pathologies believed to be caused or exacerbated by lipid peroxidation and/or free radicals, such as pathologies associated with oxidative stress, including normal aging. See Patent Publication US 2004/0092455 A1. The c compositions may also be used to treat movement disorders including restless leg syndrome, myoclonus, dystonia and/or essential tremor. See Frucht et al, Movement Disorders, 20(10), 1330 (2005).

As described herein, an oxybate-PEG ester composition may be dosed orally once per day at bedtime, e.g., between 10 pm-12 pm. This is particularly well suited for treatment of narcolepsy. Optionally, smaller doses may be delivered at bedtime and at different intervals during the night, or in the morning and at intervals during the day. Other variations may be selected depending upon the patient and the indication being treated (e.g., fibromyalgia, etc).

In one embodiment particularly well suited for treatment of narcolepsy, total oxybate in the composition is equivalent to about 4.5 to about 9 grams sodium oxybate. In certain embodiments, the composition provides a therapeutic effect for about 3.5 to about 8 hours.

As used herein, the term “powder” is a plurality of “particles” or “granules”. In certain embodiments, each particles or granules is a subunit which contain one immediate release component and/or at least one modified release component. In other embodiments, the particles or granules to contain a separate immediate release oxybate component and a separate modified release oxybate component, which are admixed. In certain embodiments, a suitable oxybate pharmaceutically acceptable salt may be included in amount up to about 10% w/w of the total oxybate content of the composition.

A “dissolution rate” refers to the quantity of drug released in vitro from a dosage form per unit time into a release medium. In vitro dissolution rates in the studies described herein were performed on dosage forms placed in a USP Type II or USP type 7 dissolution apparatus set to 37° C.±2° C. under suitable experimental conditions; see, e.g., US2012/007685, incorporated by reference herein. The dissolution media may be purified water, 0.1 N HCl, simulated gastric or intestinal fluid, or other media known in the art.

Unless as expressly stated to the contrary, the doses and strengths of oxybate are expressed in equivalent-gram (g) weights of sodium oxybate. When considering a dose of oxybate other than the sodium salt of oxybate, one must convert the recited dose or strength from sodium oxybate to the oxybate under evaluation. As used herein, the term “equivalent” to sodium oxybate is used to refer to the weight of the oxybate portion of the prodrug or conjugate, without taking into account the weight of the cyclodextrin or any matrix or coating component.

As used herein in reference to numeric values provided herein, the term “about” may indicate a variability of as much as 10%.

The following examples are illustrative only and do not limit the scope of the invention.

### Example 1: Single Cleavage Prodrugs of Oxybate

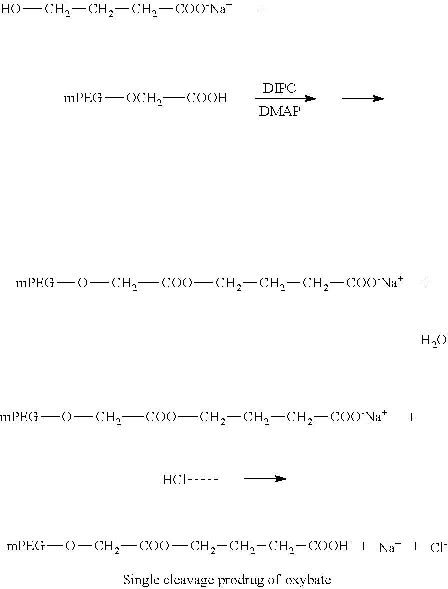

Sodium oxybate is treated mPEG-acid in presence of DIPC and DMAP. The resulting product is exposed to acidic medium to obtain single cleavage prodrug of oxybate.

### Example 2: Single Cleavage Prodrugs of Oxybate

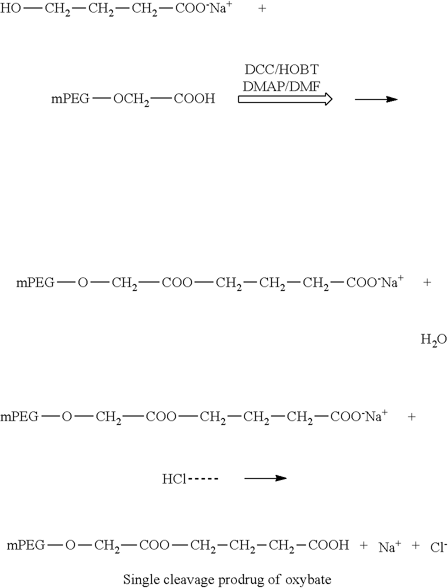

Sodium oxybate is treated with alginic acid at about 0 degree Celsius in presence of in presence of DCC/HOBT, DMAP/DMF. The resulting product is exposed to acidic medium to obtain single cleavage prodrug of oxybate.

### Example 3 Double Cleavage Di Ester Prodrugs of Oxybate

Step I

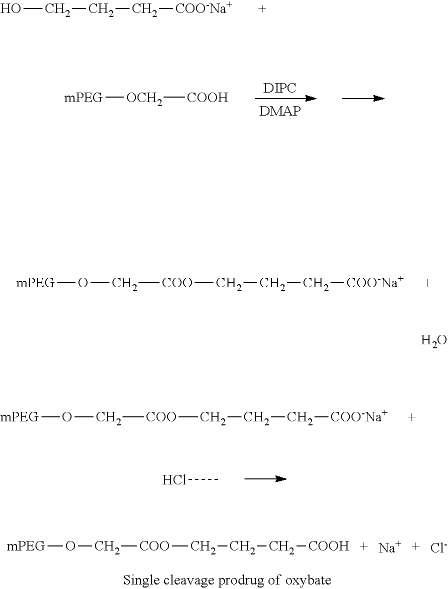

Sodium oxybate is treated mPEG-acid in presence of DIPC and DMAP. The resulting product is exposed to acidic medium to obtain single cleavage prodrug of oxybate.

Step II

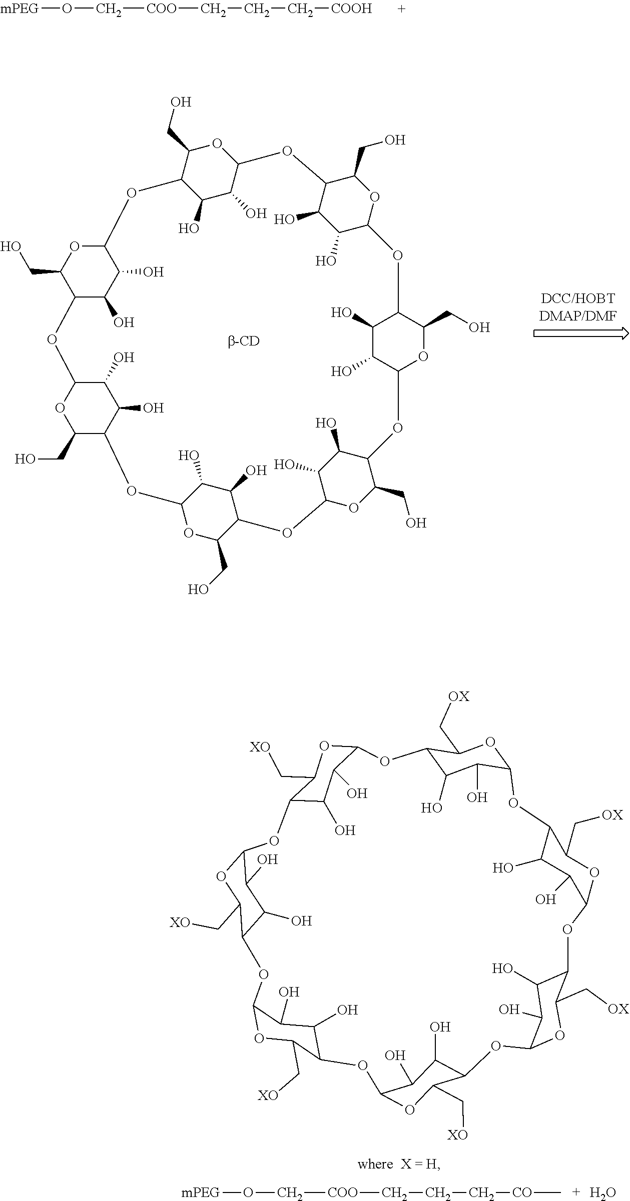

where X=H, mPEG-O—CH₂—COO—CH₂—CH₂—CH₂—CO-+H₂O

### Double Cleavage Prodrug of Oxybate

Single cleavage oxybate prodrug of step I is treated with beta-cyclodextrin at about 0 degree Celsius in presence of DCC/HOBT, DMAP/DMF to obtain mixture of 1 to 7 double cleavage prodrugs of oxybate.

### Example 4: Double Cleavage Di Ester Prodrugs of Oxybate

Step I

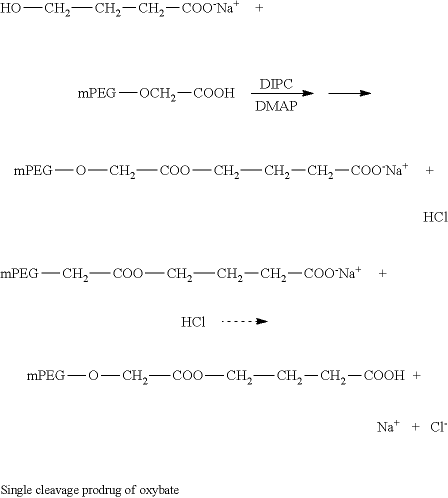

Sodium oxybate is treated mPEG-acid in presence of DIPC and DMAP. The resulting product is exposed to acidic medium to obtain single cleavage prodrug of oxybate.

Step II: Double Cleavage Prodrug of Oxybate

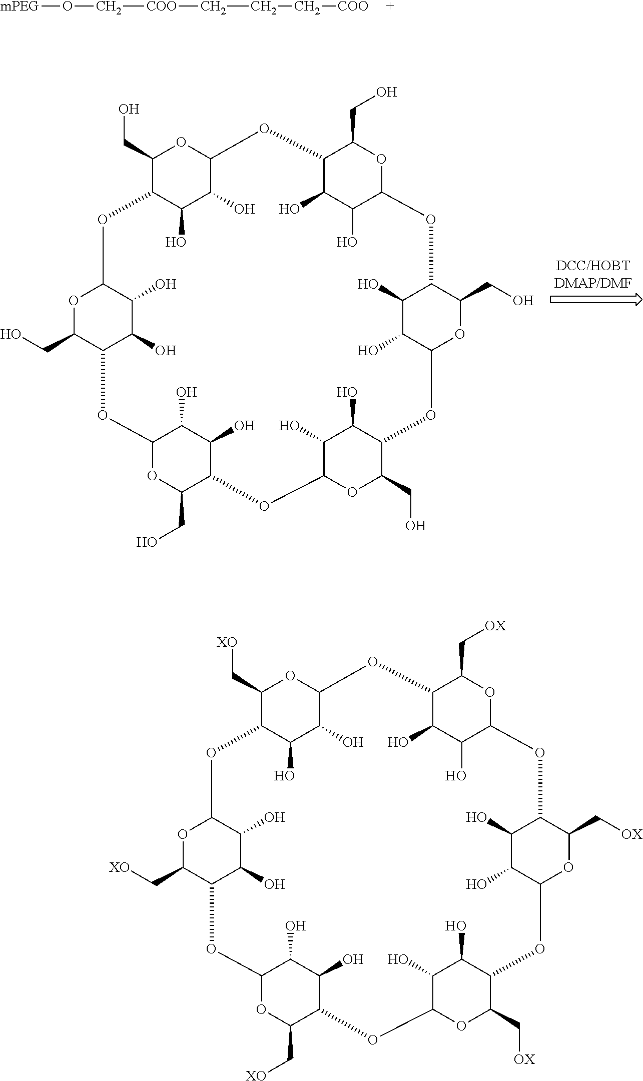

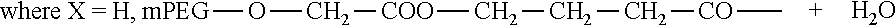

Single cleavage oxybate prodrug of step I is treated with alpha-cyclodextrin at about 0 degree Celsius in presence of DCC/HOBT, DMAP/DMF to obtain mixture of 1 to 6 double cleavage prodrugs of oxybate.

### Example 5: Double Cleavage Di Ester Prodrugs of Oxybate

Step I

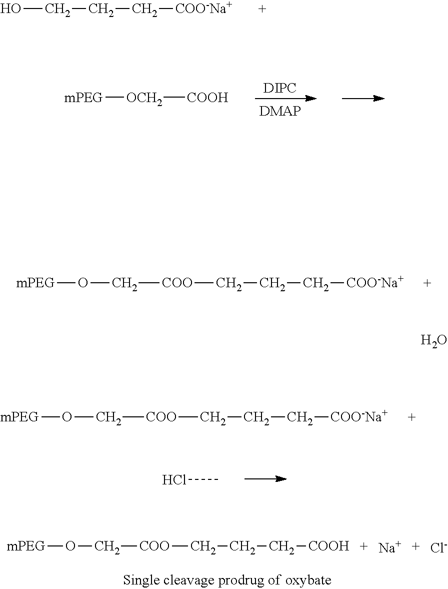

Sodium oxybate is treated mPEG-acid in presence of DIPC and DMAP. The resulting product is exposed to acidic medium to obtain single cleavage prodrug of oxybate.

Step II

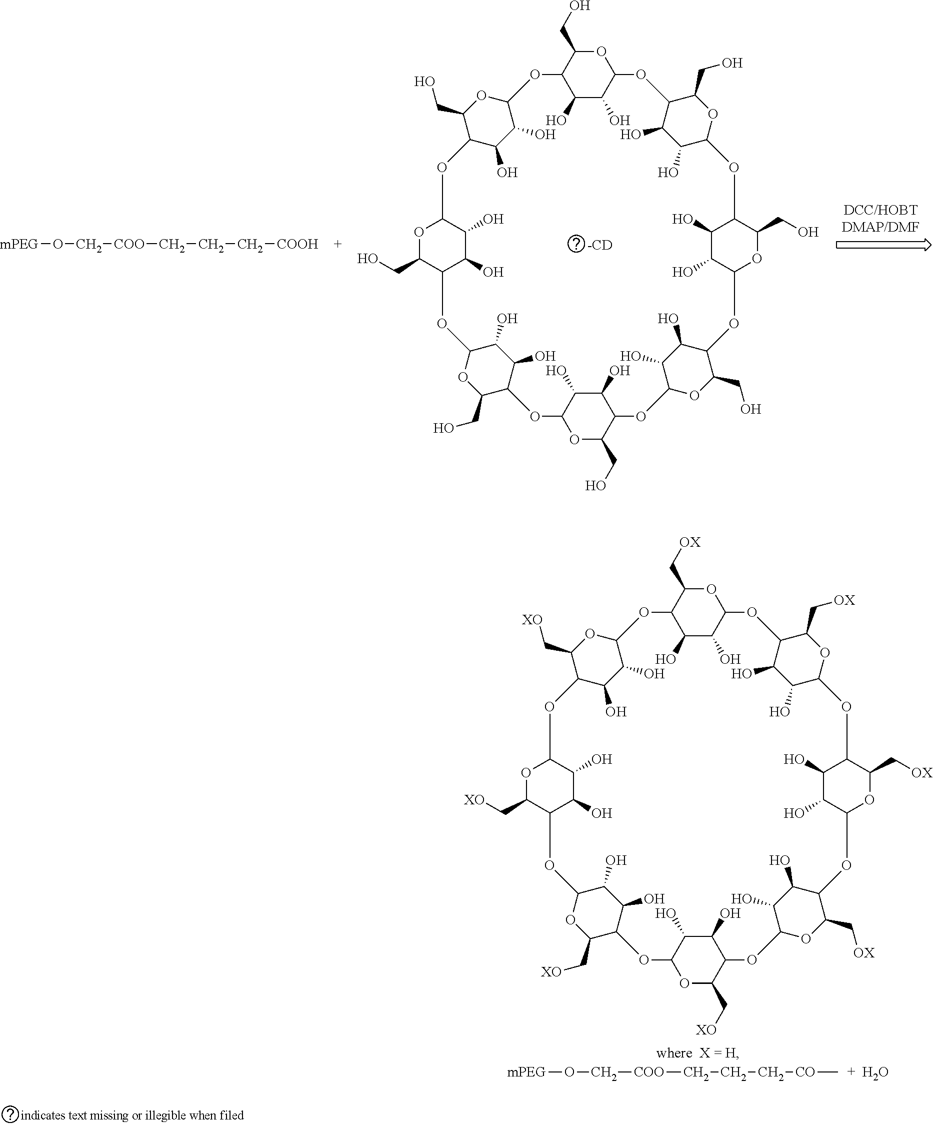

Single cleavage oxybate prodrug of step I is treated with gamma-cyclodextrin at about 0 degree Celsius in presence of DCC/HOBT, DMAP/DMF to obtain mixture of 1 to 8 double cleavage prodrugs of oxybate.

### Example 6: Double Cleavage Prodrugs of Oxybate Based on Esterification and Anion Exchange

Step I: Single Cleavage Prodrug

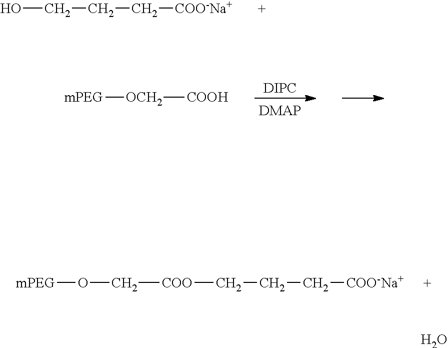

Sodium oxybate is treated mPEG-acid in presence of DIPC and DMAP to obtain single cleavage prodrug of oxybate.

Step II: Double Cleavage Prodrug

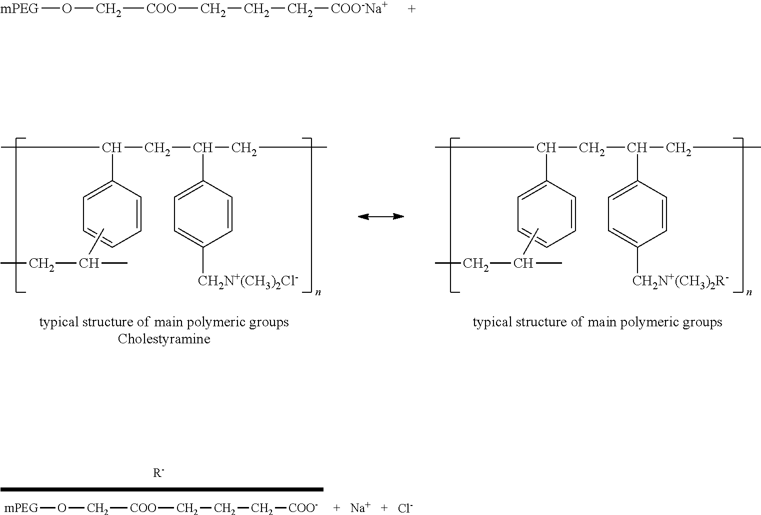

Cholestyramine is added to solution containing single cleavage prodrug of oxybate under continuous stirring. Stirring is continued further for a period of 3 hours. Double cleavage prodrug of oxybate is separated by filtration and dried.

Alternatively, oxybate cholestyramine anion exchange complex can be prepared first and then free hydroxyl group of oxybate in the ion exchange complex can be esterified with carboxylic acid group of methoxy PEG acid (mPEG acid).

### Example 7: Double Cleavage Di Ester Prodrugs of Oxybate Containing Oxybate, PEG and Beta-Cyclodextrin: Alternate Route of Synthesis

Step I

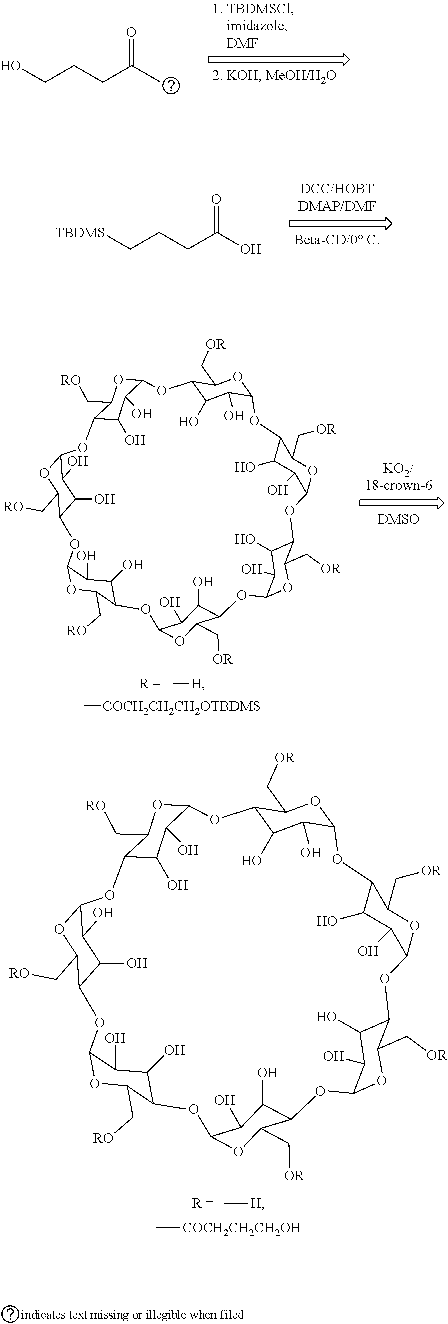

Sodium oxybate is treated with TBDMSCl, Imidazole and DMF. This is followed by treatment with KOH, Methanol and water. TBDMS substituted oxybate is obtained. TBDMS substituted oxybate is treated with beta-cyclodextrin at about 0 degree Celsius in presence of DCC/HOBT, DMAP/DMF.

The resulting product is treated with KO₂/18-crown-6 and DMSO to obtain mixture of 1 to 7 ester prodrugs of oxybate.

Step II

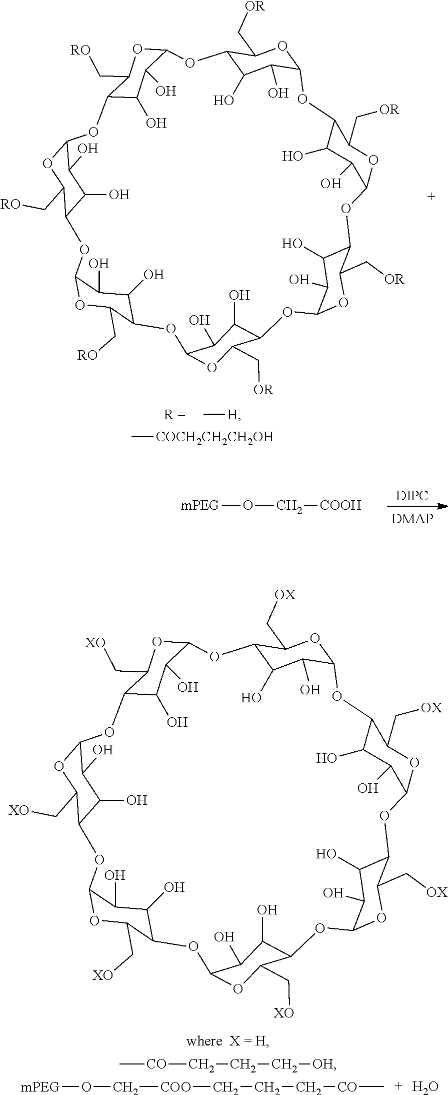

Double Cleavage Prodrugs of Oxybate

Single cleavage prodrugs of step I are treated with methoxy PEG acid in presence of DIPC and DMAP to obtain mixture of di ester prodrugs.

In mixture of di ester prodrugs formed there are different die ester prodrugs as below:

Di ester prodrugs wherein one of oxybate/s attached to beta-cyclodextrin is esterified with methoxy PEG acid,

Di ester prodrugs wherein two of oxybates attached to beta-cyclodextrin are esterified with methoxy PEG acid,

Di ester prodrugs wherein three of oxybates attached to beta-cyclodextrin are esterified with methoxy PEG acid,

Di ester prodrugs wherein four of oxybates attached to beta-cyclodextrin are esterified with methoxy PEG acid,

Di ester prodrugs wherein five of oxybates attached to beta-cyclodextrin are esterified with methoxy PEG acid,

Di ester prodrugs wherein six of oxybates attached to beta-cyclodextrin are esterified with methoxy PEG acid,

Di ester prodrugs wherein seven of oxybates attached to beta-cyclodextrin are esterified with methoxy PEG acid.

### Example 8: Double Cleavage Di Ester Prodrugs Containing Oxybate, PEG and Polymerized Beta-Cyclodextrin

Sodium oxybate is treated mPEG-acid in presence of DIPC and DMAP. The resulting product is exposed to acidic medium to obtain single cleavage prodrug of oxybate. Single cleavage oxybate prodrug of step I is treated with polymerized beta-cyclodextrin at about “0” degree Celsius in presence of DCC/HOBT, DMAP/DMF to obtain mixture of 1 to “n” double cleavage prodrugs of oxybate.

All patents, patent publications, and other publications listed in this specification, as well as U.S. Patent Application No. 63/231,878, filed Aug. 11, 2021, are incorporated herein by reference. While the invention has been described with reference to a particularly preferred embodiment, it will be appreciated that modifications can be made without departing from the spirit of the invention. Such modifications are intended to fall within the scope of the appended claims.

1. A oxybate polyethylene glycol (PEG) ester prodrug or conjugate, said prodrug or conjugate comprising:
(A) an oxybate-PEG ester having the structure: PEG-O—CH₂—COO—CH₂—CH₂—CH₂—COOH, wherein PEG is bound to oxybate through an ester linkage formed between the gamma hydroxyl group of oxybate and carboxylic acid group of an Alkoxy-PEG acid;
(B) a PEG-oxybate-cyclodextrin di-ester, wherein PEG is bound to oxybate through an ester linkage formed between the gamma hydroxyl group of oxybate and carboxylic acid group of an Alkoxy PEG acid and cyclodextrin is bound to the oxybate through another ester linkage formed between carboxylic acid group of oxybate and hydroxyl group of cyclodextrin or
(C) a PEG-oxybate-anion exchange resin complex, wherein PEG is bound to oxybate through an ester linkage formed between the gamma hydroxyl group of oxybate and carboxylic acid group of an Alkoxy PEG acid and the anion exchange resin is bound through ionic bond formed between quaternary ammonium group of the resin and carboxylic acid group of oxybate, the complex is optionally in a polymeric matrix, and/or optionally coated.

2. The oxybate-PEG ester of claim 1, wherein the ester has the structure of (A).

3. The oxybate-PEG ester of claim 1, wherein the prodrug or conjugate of (B) has the structure of:
(A) an PEG-oxybate alpha-cyclodextrin polymer di-ester of Formula IA: 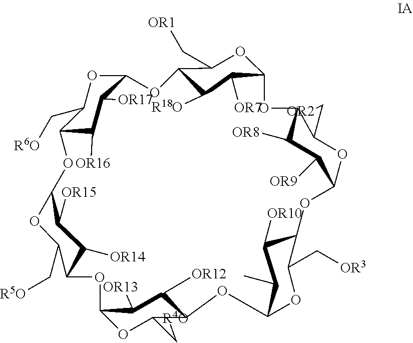
wherein in Formula IA, R¹-R¹⁸ are independently selected from —H, —COCH₂CH₂CH₂OH; or PEG-O—CH₂—COO—CH₂—CH₂—CH₂—CO— to form an PEG-oxybate alpha-cyclodextrin ester, provided that at least one of R¹⁻¹⁸ is PEG-O—CH₂—COO—CH₂—CH₂—CH₂—CO—;
(B) an PEG-oxybate beta-cyclodextrin polymeric ester of Formula IB: 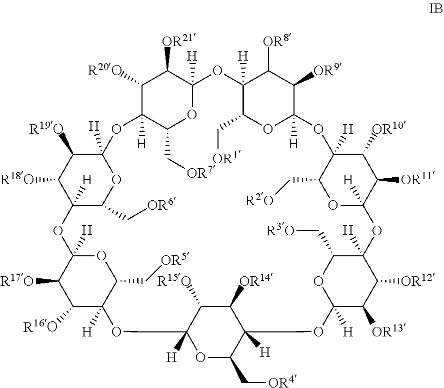
wherein in Formula IB, R^{1′}-R^{21′} are independently —H, —COCH₂CH₂CH₂OH or -PEG-O—CH₂—COO—CH₂—CH₂—CH₂—CO— to form an PEG-oxybate beta-cyclodextrin di-ester,
provided that at least one of R′-R^{21′} is -PEG-O—CH₂—COO—CH₂—CH₂—CH₂—CO—; or
(C) an oxybate-PEG gamma-cyclodextrin ester of Formula IC: 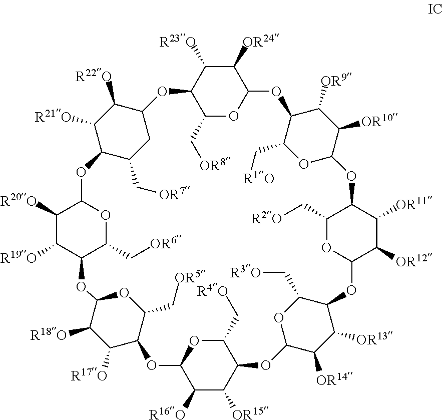
wherein in Formula IC, R^{1″}-R^{24″} are independently selected from —H, —COCH₂CH₂CH₂OH or PEG-O—CH₂—COO—CH₂—CH₂—CH₂—CO— to form an oxybate-PEG gamma-cyclodextrin ester, provided that at least one of R¹-R^{24″} is PEG-O—CH₂—COO—CH₂—CH₂—CH₂—CO—.

4. The oxybate-PEG ester of claim 3, wherein the cyclodextrin is a beta-cyclodextrin.

5. The oxybate-PEG ester of claim 3, wherein the cyclodextrin is an alpha-cyclodextrin.

6. The oxybate-PEG ester of claim 3, wherein the cyclodextrin is a gamma-cyclodextrin.

7. The oxybate-PEG ester of claim 1, which is (C) and the anion exchange resin is a cholestyramine.

8. The oxybate-PEG ester of claim 1, wherein the Alkoxy PEG is a C1-C4 Alkoxy.

9. The oxybate-PEG ester of claim 8, wherein the Alkoxy PEG is a methyl PEG.

10. A pharmaceutical composition comprising oxybate-polyethylene glycol (PEG) prodrug or conjugates which comprises:
(A) an oxybate-PEG ester having the structure: PEG-O—CH₂—COO—CH₂—CH₂—CH₂—COOH, wherein PEG is bound to oxybate through an ester linkage formed between the gamma hydroxyl group of oxybate and carboxylic acid group of an Alkoxy-PEG acid;
(B) a PEG-oxybate-cyclodextrin di-ester, wherein PEG is bound to oxybate through an ester linkage formed between the gamma hydroxyl group of oxybate and carboxylic acid group of an Alkoxy PEG acid and cyclodextrin is bound to the oxybate through another ester linkage formed between carboxylic acid group of oxybate and hydroxyl group of cyclodextrin; or
(C) a PEG-oxybate-anion exchange resin complex, wherein PEG is bound to oxybate through an ester linkage formed between the gamma hydroxyl group of oxybate and carboxylic acid group of an Alkoxy PEG acid and the anion exchange resin is bound through ionic bond formed between quaternary ammonium group of the resin and carboxylic acid group of oxybate, the complex is optionally in a polymeric matrix, and/or optionally coated.

11. The pharmaceutical composition of claim 10, further comprising: one or more oxybate-PEG esters.

12. The pharmaceutical composition of claim 10, wherein the prodrug or conjugate of (B) has the structure of:
(A) an PEG-oxybate alpha-cyclodextrin polymer di-ester of Formula IA: 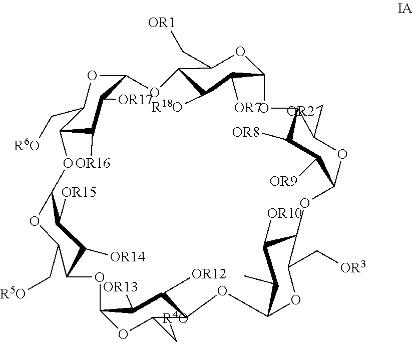
wherein in Formula IA, R¹-R⁸ are independently selected from —H, —COCH₂CH₂CH₂OH; or PEG-O—CH₂—COO—CH₂—CH₂—CH₂—CO— to form an PEG-oxybate alpha-cyclodextrin di-ester, provided that at least one of R¹⁻¹⁸ is —HOCH₂CH₂CH₂O-PEG;
(B) an PEG-oxybate beta-cyclodextrin di-ester of Formula IB: 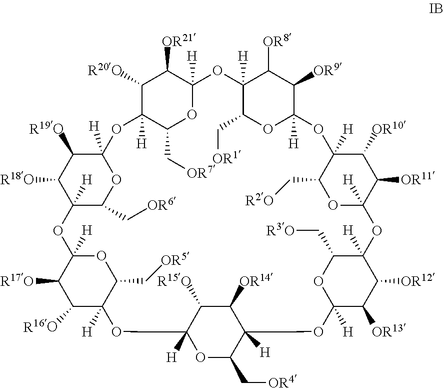
wherein in Formula IB, R^{1′}-R^{21′} are independently —H, —COCH₂CH₂CH₂OH or -PEG-O—CH₂—COO—CH₂—CH₂—CH₂—CO— to form an PEG-oxybate beta-cyclodextrin di-ester,
provided that at least one of R′-R^{21′} is PEG-O—CH₂—COO—CH₂—CH₂—CH₂—CO—; or
(C) an oxybate-PEG gamma-cyclodextrin ester of Formula IC: 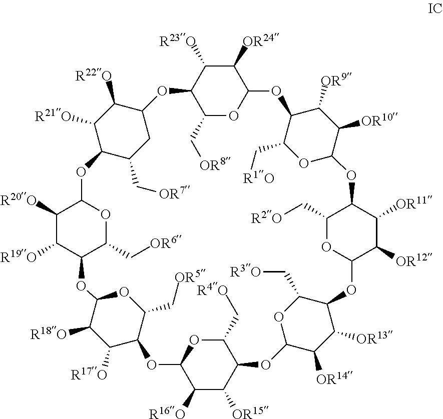
wherein in Formula IC, R^{1″}-R^{24″} are independently selected from —H, —COCH₂CH₂CH₂OH or PEG-O—CH₂—COO—CH₂—CH₂—CH₂—CO— to form an oxybate-PEG gamma-cyclodextrin ester, provided that at least one of R¹-R^{24″} is PEG-O—CH₂—COO—CH₂—CH₂—CH₂—CO—

13. The pharmaceutical composition of claim 12, wherein the cyclodextrin is a beta-cyclodextrin.

14. The pharmaceutical composition of claim 12, wherein the cyclodextrin is an alpha-cyclodextrin.

15. The pharmaceutical composition of claim 12, wherein the cyclodextrin is a gamma-cyclodextrin.

16. The pharmaceutical composition of claim 12, which is (C) and the anion exchange resin is a cholestyramine.

17. The pharmaceutical composition of claim 16, wherein the PEG-oxybate-anion exchange resin complex is in a matrix further comprising a hydrophilic or hydrophobic granulating agent, and a modified release barrier coating is over the PEG-oxybate-anion exchange resin complex-matrix particles.

18. The pharmaceutical composition of claim 17, which further comprises an immediate release PEG-oxybate-anion exchange resin complex.

19. The pharmaceutical composition of claim 17, which further comprises an immediate release PEG-oxybate-anion exchange resin complex.

20. The pharmaceutical composition of claim 17, which further comprises an immediate release PEG-oxybate-anion exchange resin complex.

21. The pharmaceutical composition of claim 10, which further comprises one or more pharmaceutically acceptable oxybate salts.

22. The pharmaceutical composition of claim 10, wherein the composition comprises an amount of oxybate equivalent of 2.25 gm oxybate HCl to 12 gm sodium oxybate.

23. The pharmaceutical composition of claim 10, wherein the pharmaceutical composition is a powder-for-suspension or powder-for-solution.

24. The pharmaceutical composition of claim 10, wherein the pharmaceutical composition is an aqueous suspension or solution formulated for injection.

25. The pharmaceutical composition of claim 10, wherein the pharmaceutical composition is an aqueous suspension or solution formulated for oral dosing.

26. The pharmaceutical composition of claim 10, wherein the pharmaceutical composition is sodium-free.

27. The pharmaceutical composition of claim 10, wherein the composition comprises one or more oxybate-PEG compounds of (A), (B) and/or (C) in both immediate release and extended release forms.

28. A method for improving the abuse-resistance of an oxybate composition comprising generating an oxybate cyclodextrin ester of claim 1.
